(19) United States
(12) Patent Application Publication
(10) Pub. No.: US 2020/0390596 A1
(43) Pub. Date: Dec. 17, 2020

(54) Therapeutic Agent Delivery Devices Having Integrated Pain Mitigation, and Methods for Using the Same

(71) Applicant: iRenix Medical, Inc., Palo Alto, CA (US)

(72) Inventors: Thomas W. Chalberg, Jr., Palo Alto, CA (US); Espir Gabriel Kahatt, Carlsbad, CA (US); Victor W. Chang, San Diego, CA (US); Stephen J. Smith, Palo Alto, CA (US)

(21) Appl. No.: 16/772,565

(22) PCT Filed: Jan. 3, 2019
(86) PCT No.: PCT/US2019/012202

§ 371 Date: Jun. 12, 2020

Related U.S. Application Data
(60) Provisional application No. 62/614,248, filed on Jan. 5, 2018.
(60) Provisional application No. 62/613,324, filed on Jan. 3, 2018.

(51) Int. Cl. A61F 9/00; A61K 39/395; A61K 38/17

(52) U.S. Cl. CPC A61F 9/0008; A61K 38/1793; A61K 39/3955

(57) ABSTRACT

Therapeutic agent delivery devices having integrated pain mitigation are provided. Aspects of the devices include a therapeutic agent delivery system; and an actuator component, where the therapeutic agent delivery system is present in a receiving space of the actuator component. The therapeutic agent delivery system includes dmg container, a needle and a tissue contacting tip. The actuator component includes a therapeutic agent delivery system actuator and a pain mitigation system. Also provided are methods of using the devices, as well as kits that include various components of the systems.

### CROSS-REFERENCE TO RELATED APPLICATIONS

This application is related to U.S. Provisional Patent Application Ser. No. 62/613,324 filed Jan. 3, 2018 and U.S. Provisional Patent Application Ser. No. 62/614,248 filed Jan. 5, 2018; the disclosures of which applications are herein incorporated by reference.

### INTRODUCTION

Pain is a major limiting factor in many common procedures performed in the inpatient and ambulatory care settings. A very abbreviated list of such procedures includes skin biopsy, fine needle aspiration biopsy, IV insertion, vaccination, injections (including injection of anesthetics and gasses), blood draws, central line placements, and finger and heal pricks for blood analysis (glucose measurement). Pharmacologic anesthesia is a primary method of pain reduction, but the delivery of local pharmacologic anesthesia usually requires a painful injection.

The ocular surface is a tissue surface to which therapeutic agents may be delivered. The ability to deliver medication directly into the eye via intravitreal injection therapy (IVT) has transformed the treatment landscape of a number of previously blinding diseases, including macular degeneration and diabetic retinopathy. The success of these therapies in preventing blindness has resulted in a dramatic increase in the number of intravitreal injections performed, with an estimated 4.1 million injections given in the United States alone in 2013. The number of indications for IVT continues to expand, increasing utilization of this therapy significantly every year. The primary limitations of IVT are patient discomfort, ocular surface bleeding, corneal toxicity, and the time constraints of treating the vast number of patients requiring this therapy. These drawbacks relate to the difficulty of delivering ocular anesthesia to the highly vascularized ocular surface.

To give an ocular injection, the physician first provides ocular surface anesthesia by one or more of a number of methods, including the following: topical application of anesthetic drops; a subconjunctival injection of lidocaine; placement of cotton tipped applicators (commonly called a “pledget”) soaked in lidocaine over the planned injection site, application of topical anesthetic gel, or some combination of these. Following ocular anesthesia, the physician or an assistant sterilizes the periocular region by coating it in betadine or a similar antiseptic. Optionally, an eyelid speculum is placed, and the physician marks the location of the injection using calipers that guide placement of the needle. The ocular surface is again sterilized, and the physician gives the injection. Current methods of local anesthesia have unique drawbacks and patients often experience discomfort during and after intraocular injections.

### SUMMARY

Therapeutic agent delivery devices having integrated pain mitigation are provided. Aspects of the devices include a therapeutic agent delivery system and an actuator component, where the therapeutic agent delivery system is present in a receiving space of the actuator component. The therapeutic agent delivery system includes drug container, a needle and a tissue contacting tip. The actuator component includes a therapeutic agent delivery system actuator and a pain mitigation system. Also provided are methods of using the devices, as well as kits that include various components of the systems.

### BRIEF DESCRIPTION OF THE FIGURES

FIG. 1 provides a view of a hand held therapeutic agent delivery device according to an embodiment of the invention.

FIG. 2 provides a cutaway view of the device shown in FIG. 1.

FIG. 3 provides various views of a therapeutic agent delivery system and components thereof, according to an embodiment of the invention.

FIG. 4 provides a view of a cooling system of the device shown in FIGS. 1 and 2.

FIG. 5 provides a view of a hand held therapeutic agent delivery device according to an embodiment of the invention.

FIG. 6 provides a cutaway view of the device shown in FIG. 5.

FIGS. 7A and 7B provide views of a composite tip/needle according to an embodiment of the invention.

FIGS. 8A to 8C provide views of a hand held therapeutic agent delivery device according to an embodiment of the invention.

FIG. 9 provides a view docking station according to an embodiment of the invention.

FIG. 10 provides further details regarding a smart device system of the invention.

### DEFINITIONS

As used herein, the term “tissue” refers to one or more aggregates of cells in a subject (e.g., a living organism, such as a mammal, such as a human) that have a similar function and structure or to a plurality of different types of such aggregates. Tissue may include, for example, organ tissue, muscle tissue (e.g., cardiac muscle; smooth muscle; and/or skeletal muscle), connective tissue, ocular conjunctival tissue, nervous tissue and/or epithelial tissue.

The term “subject” is used interchangeably in this disclosure with the term “patient”. In certain embodiments, a subject is a “mammal” or “mammalian”, where these terms are used broadly to describe organisms which are within the class mammalia, including the orders carnivore (e.g., dogs and cats), rodentia (e.g., mice, guinea pigs, and rats), and primates (e.g., humans, chimpanzees, and monkeys). In some embodiments, subjects are humans. The term “humans” may include human subjects of both genders and at any stage of development (e.g., fetal, neonates, infant, juvenile, adolescent, adult), where in certain embodiments the human subject is a juvenile, adolescent or adult. While the devices and methods described herein may be applied to perform a procedure on a human subject, it is to be understood that the subject devices and methods may also be carried out to perform a procedure on other subjects (that is, in “non-human subjects”).

In some instances, the devices or portions thereof may be viewed as having a proximal and distal end. The term “proximal” refers to a direction oriented toward the operator during use or a position (e.g., a spatial position) closer to the operator (e.g., further from a subject or tissue thereof) during use (e.g., at a time when a tissue piercing device enters tissue). Similarly, the term “distal” refers to a direction oriented away from the operator during use or a position (e.g., a spatial position) further from the operator (e.g., closer to a subject or tissue thereof) during use (e.g., at a time when a tissue piercing device enters tissue). Accordingly, the phrase “proximal end” refers to that end of the device that is closest to the operator during use, while the phrase “distal end” refers to that end of the device that is most distant to the operator during use.

Modules are made up of one or more functional blocks which act in concert to perform a particular function, which is the purpose of the module. A given module may be implemented as hardware, software or a combination thereof. In some instances, modules may include a circuitry element, such as an integrated circuit. When present, integrated circuits may include a number of distinct functional blocks, where the functional blocks are all present in a single integrated circuit on an intraluminal-sized support. By single integrated circuit is meant a single circuit structure that includes all of the different functional blocks. As such, the integrated circuit is a monolithic integrated circuit (also known as IC, microcircuit, microchip, silicon chip, computer chip or chip) that is a miniaturized electronic circuit (which may include semiconductor devices, as well as passive components) that has been manufactured in the surface of a thin substrate of semiconductor material.

Furthermore, the definitions and descriptions provided in one or more (e.g., one, two, three, or four, etc.) sections of this disclosure (e.g., the “Descriptions”, “Devices”, “Methods” and/or “Kits” sections below) are equally applicable to the devices, methods and aspects described in the other sections.

### DETAILED DESCRIPTION

Therapeutic agent delivery devices having integrated pain mitigation are provided. Aspects of the devices include a therapeutic agent delivery system and an actuator component, where the therapeutic agent delivery system is present in a receiving space of the actuator component. The therapeutic agent delivery system includes drug container, a needle and a tissue contacting tip. The actuator component includes a therapeutic agent delivery system actuator and a pain mitigation system. Also provided are methods of using the devices, as well as kits that include various components of the systems.

Before the present invention is described in greater detail, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

While the apparatus and method has or will be described for the sake of grammatical fluidity with functional explanations, it is to be expressly understood that the claims, unless expressly formulated under 35 U.S.C. § 112, are not to be construed as necessarily limited in any way by the construction of “means” or “steps” limitations, but are to be accorded the full scope of the meaning and equivalents of the definition provided by the claims under the judicial doctrine of equivalents, and in the case where the claims are expressly formulated under 35 U.S.C. § 112 are to be accorded full statutory equivalents under 35 U.S.C. § 112.

### Devices

As summarized above, therapeutic agent delivery devices having integrated pain mitigation are provided. As the devices are therapeutic agent delivery devices, they are configured to deliver an amount (e.g., dosage) of a therapeutic agent to a target tissue delivery site of a subject. As will be reviewed in greater detail below, the therapeutic agent may be in any convenient state, such as liquid, solid or semi-solid (e.g., gel), or gas. In some instances, the devices deliver a liquid active agent composition to a target deliver site. As the devices have integrated pain mitigation, they are configured to alleviate pain associated with delivery of the therapeutic agent to the target tissue delivery site by the device. While the magnitude of pain mitigation may vary, in some instances the magnitude of pain mitigation is 5% or more, such as 10% or more, and including 20% or more, as compared to a suitable control (such as identical delivery without pain mitigation).

Aspects of the devices include a therapeutic agent delivery system and an actuator component, where the therapeutic agent delivery system is present in a receiving space of the actuator component and the actuator component includes a therapeutic agent delivery system actuator. Aspects of the devices further include a pain mitigation system in the actuator component. In some instances, the therapeutic agent delivery system is release-ably engaged in the receiving space of the actuator component. Accordingly, in such instances the therapeutic agent delivery system is configured to be readily separable from the receiving space of the actuator component without in any way damaging the functionality of the actuator component, such that another therapeutic agent delivery system may be positioned in the receiving space of the actuator component. As such, the devices of the present invention are configured so that the actuator component can be sequentially employed with multiple different therapeutic agent delivery systems. Of interest are configurations in which the therapeutic agent delivery system can be manually operably positioned in the receiving space of the actuator component unit without the use of any tools. In some instances, the device further includes a locking element for release-ably engaging the therapeutic agent delivery system in the receiving space of the actuator component of the device. Any convenient locking mechanism may be employed, such as but not limited to: press fit (e.g., in the form of ridges and corresponding notches, a clip in the receiving space configured to press fit engage), mechanical, e.g., a movable arm that moves between a first position that holds the delivery system in the delivery space and a second position that does not, and the like.

As reviewed above, the therapeutic delivery devices include an integrated pain mitigation system. A pain mitigation system is a system that provides for pain alleviation during delivery of a therapeutic agent to a target delivery site, as discussed above. The pain mitigation system may vary as desired, where pain mitigation systems finding use in devices of the invention include both anesthesia producing systems (i.e., systems that result in at least some degree of, if not complete loss of, sensation in the target tissue delivery site, e.g., via blockage of all feeling in the target tissue delivery site) and analgesia producing systems (i.e., systems that result in relief of pain without total loss of feeling in the target tissue delivery site).

In some instances, an anesthesia producing system is a cooling system, i.e., a system that decreases the temperature of the target tissue delivery site by an amount sufficient to produce the desired anesthesia in the target tissue delivery site. The cooling system may vary, and in some instances is a system that provides for contact of a cold element (e.g., a cold tip or cold tissue engager (such as a tissue contacting tip, e.g., as described in greater detail below) with the target tissue delivery site. The cold element (which may be a component of a tissue engager, e.g., as described in greater detail below) of the cooling system may vary, and in some instances is an element that is configured to maintain a temperature of between −80° C. to +5° C., such as −20° C. to 0° C. and including −10° C. to −5° C. when contacted with the target tissue delivery site. During a given delivery method, a tissue engager may maintain a constant temperature or cycle through one or more distinct temperature ranges, as desired. For example, a tissue engager may be configured to have a temperature that falls within a first range (e.g., as described above) to provide for desired cryoanesthesia during therapeutic agent delivery, and then cycle to a second, warmer temperature prior to remove of the device, such as a temperature ranging from 0 to −5, such as 0 to −2.5, including 0 to −1° C. Where the target tissue delivery site is an ocular tissue delivery site, e.g., as described elsewhere, delivery of cooling to cause rapid vasoconstriction enables a reduction in the occurrence of ocular surface bleeding and prevents repeated vascular trauma with long term circulatory compromise.

Specific cooling systems of interest that may find use in anesthesia producing pain mitigation systems may vary, where cooling systems of interest include, but are not limited to: thermoelectric cooling systems, liquid evaporation cooling systems, solid sublimation cooling systems, Joule-Thompson cooling systems, thermodynamic cycle cooling systems, endothermic reaction cooling systems and low-temperature substance cooling systems, and the like.

In some instances, the pain mitigation anesthesia producing system is a thermoelectric cooling system, e.g., one that includes one or a combination of thermoelectric (Peltier) devices or units. While thermoelectric cooling systems employed in embodiments of devices of the invention may vary, in some instances the thermoelectric cooling systems include a cold tip that is configured to contact a target tissue delivery site (and therefore may also be referred to as a tissue engager), as well as one or more of a power source, a controller, a cooling power concentrator, one or more Peltier unit modules, and a heat sink (which may be a solid material or include one a fluid, such as a liquid, phase in a container. It should be understood that, in some embodiments, a given thermoelectric cooling system may include a heating element (not shown) that operates in conjunction with the cooling elements to precisely maintain a desired temperature and/or heat flux. Further details regarding embodiments of thermoelectric cooling systems that may be employed in devices of the invention are provided in U.S. Published Patent Application Publication No. 20160279350; the disclosure of which is herein incorporated by reference.

In yet other instances, the cooling system may include a substance having a freezing temperature of 0° C. or lower.

As reviewed above, other non-thermoelectric cooling pain mitigation systems may be employed, such as but not limited to: liquid evaporation cooing system, solid sublimation cooling system, Joule-Thompson cooling system, thermodynamic cycle cooling system, an endothermic reaction cooling system and a low-temperature substance cooling system.

Instead of cooling systems, other types of anesthesia producing systems may be employed as pain mitigation systems. Such anesthesia systems include, but are not limited to: system that deliver an anesthetic agent, such as but not limited to: sodium-channel blockers, e.g., as amino amides or amino esters, i (such as proparacaine, tetracaine, or lidocaine drops, gels, or creams), naturally-derived agents, such as saxitoxin, neosaxitoxin, tetrodotoxin, menthol, eugenol, and cocaine; and the like.

Also of interest as pain mitigation systems are analgesia producing systems, e.g., as summarized above. Examples of analgesia producing systems finding use in embodiments of devices of the invention include application of agents considered above as local anesthetics. They may also include, but are not limited to, additional techniques such as electrical stimulation (Campbell and Taub, Arch Neurol. 1973; 28(5):347-350.) and the like.

Therapeutic agent delivery devices as described herein may be handheld. In such embodiments, as the devices are handheld, they are configured to be held easily in the hand of an adult human. Accordingly, the devices may have a configuration that is amenable to gripping by the human adult hand. The weight of the devices may vary, and in some instances may range from 0.05 to 3 pounds, such as 0.1 pounds to 1 pound. Handheld devices of the invention may have any convenient configuration, where examples of suitable handle configurations are further provided below.

The therapeutic agent delivery devices of the invention may be configured for delivery of a therapeutic agent to a variety of target tissue delivery sites. Examples of target tissue delivery sites include both external and internal delivery sites, wherein internal delivery sites include those sites located in body cavities. External sites may include keratinized sites, as well as sites characterized by cutaneous membranes, mucous membranes, and tissue of the mucocutaneous zone. In some instances, the target tissue delivery site is an ocular tissue delivery site, where ocular tissue delivery sites of interest include a region that begins at the corneal limbus and extends anywhere from 1 mm to 10 mm posterior to the limbus, 2 mm to over 8 mm posterior to the limbus, such as 3 mm to 6 mm from the corneal limbus, e.g., 3 to 4 mm from the corneal limbus, e.g., to allow intraocular injection via Pars plana or Pars plicata. Ocular tissue delivery sites may include conjunctiva, episclera, and sclera of the eye. Ocular tissue delivery sites of interest include those that provide for intravitreal injection therapy (IVT), retrobulbar injection therapy, subtenon injection therapy, subretinal injection therapy, suprachorodial injection, subconjunctival injection therapy, intracameral injection therapy, and the like.

As summarized above, delivery devices of the invention include a therapeutic agent delivery system operably engaged in a receiving space of an actuator component. Each of these components of the device is now described separately in greater detail.

### Therapeutic Agent Delivery System

The therapeutic agent delivery system is configured to be operably, and in some instances release-ably, engaged in a receiving space of an actuator component of the device, e.g., as described above. The therapeutic agent delivery system is a system that, upon actuation by an actuator of the actuator component, delivers an amount, e.g., a dosage, of a therapeutic agent to a target tissue delivery site. As summarized above, the therapeutic agent composition that is delivered to the target tissue delivery site may be a composition that is in a variety of different physical states, including liquid, solid, semi-solid (e.g., gel) and gaseous. As such, the therapeutic agent delivery system may vary depending on the physical state of the therapeutic agent composition.

In some embodiments, the therapeutic agent composition is a liquid active agent composition. In such instances, the therapeutic agent delivery systems include: a drug container having a proximal and distal end and comprising a liquid active agent composition; a needle having a proximal and distal end, where the proximal end is operatively coupled to the distal end of the drug container; and a tissue contacting tip operatively coupled to the needle.

The drug container is configured to hold a desired amount of an active agent composition. While the volume of the drug container may vary, in some instances the volume ranges from 0.10 to 5.0 cc, such as 0.25 to 1.50 cc, including 0.50 to 1.0 cc, e.g., 0.70 to 0.80 cc. In some instances, the volume of the container is sufficient to hold an amount of a therapeutic agent composition that is greater than the amount which is delivered to a target tissue delivery site during use of the device. While the magnitude of the excess may vary, in some instances the magnitude ranges from 110% to 500% of the delivered volume, such as 120% to 150%. Where the therapeutic agent composition is a non-gaseous composition, the amount of any gas, e.g., air, in the reservoir (and other components of the therapeutic agent delivery system, may be minimal, where in some instances the amount is 10 μl or less, such as 5 μl or less, 3 μl or less, 2 μl or less, or 1 μl or less. Any desired active agent composition may be present in the container (i.e., reservoir). Examples of therapeutic active agents that may be present include, but are not limited to steroids such as corticosteroids including dexamethasone, fluocinolone, loteprednol, difluprednate, fluorometholone, prednisolone, medrysone, triamcinolone, betamethasone and rimexolone; nonsteroidal anti-inflammatory agents such as salicylic-, indole acetic-, aryl acetic-, aryl propionic- and enolic acid derivatives including bromfenac, diclofenac, flurbiprofen, ketorolac tromethamine and nepafenac; antibiotics including azithromycin, bacitracin, besifloxacin, ciprofloxacin, erythromycin, gatifloxacin, gentamicin, levofloxacin, moxifloxacin, ofloxacin, sulfacetamide and tobramycin; VEGF inhibitors such as tyrosine kinase inhibitors, antibodies to VEGF, antibody fragments to VEGF, VEGF binding fusion proteins; PDGF inhibitors, antibodies to PDGF, antibody fragments to PDGF, PDGF binding fusion proteins; anti-TNF alpha agents such as TNF-alpha binding agents, including antibodies to TNF-alpha, antibody fragments to TNF-alpha and TNF-alpha binding fusion proteins, including infliximab, etanercept, adalimumab, certolizumab and golimumab; mTOR inhibitors such as sirolimus, sirolimus analogues, Everolimus, Temsirolimus and mTOR kinase inhibitors; cells such as mesenchymal cells (e.g. mesenchymal stem cells), or cells transfected to produce a therapeutic compound; neuroprotective agents such as antioxidants, calcineurin inhibitors, NOS inhibitors, sigma-1 modulators, AMPA antagonists, calcium channel blockers and histone-deacetylases inhibitors; antihypertensive agents such as prostaglandin analogs, beta blockers, alpha agonists, and carbonic anhydrase inhibitors; aminosterols such as squalamine; antihistamines such as H 1-receptor antagonists and histamine H2-receptor antagonists; therapeutic cells; tyrosine kinase inhibitors and nucleic acid based therapeutics such as gene vectors, complement system modulators, e.g., inhibitors; chemotherapeutic agents; insulin; cytokines, e.g., interferon beta 1-alpha; nucleic acid active agents, e.g., plasmids and siRNA; interleukin-4 receptor antagonists (such as Dupilumab etc.), interleukin-6 receptor antagonists (such as Sarlumab, etc.), proprotein convertase subtilisin kexin type 9 inhibitor antibodies (such as Alirocumab), interleukin-1 inhibitors (such as Rilonacept), and PD-1 checkpoint inhibitors (such as Cemiplimab), monoclonal antibodies targeting HER2 (such as trastuzumab), monoclonal antibodies that target CD52 (such as Alemtuzumab), RANK/RANKL inhibition (such as denosumab), antibodies against CD20 (such as rituximab and veltuzumab), and antibodies directed against CA125 (such as abagovomab); and the like.

In some instances, the active agent composition is a vaccine composition. As described herein, a vaccine composition is a composition that includes one or more immunogens for vaccinating a mammal (e.g., a dog, cat, horse, sheep, pig, cow, heifer, calf, steer, bull, goat, llama, ferret, or human), a bird (e.g., a chicken and turkey), or a fish (e.g., a trout, salmon, or sea bass). For example, a vaccine composition can include the immunogen or immunogens or vaccine antigen or antigens of an anti-pathogen vaccine, an anti-cancer vaccine, or an immunocontraception vaccine. Examples of immunogens of an anti-pathogen vaccine that can be formulated into a vaccine composition and used as described herein include, without limitation, antigens (e.g., hemagglutinin, neuraminidases, glycoproteins, or nucleoproteins) from viruses such as dengue, HIV, influenza, HPV, HSV, HZV, feline panleukopenia virus, feline infectious peritonitis virus, rabies, porcine reproductive and respiratory disease virus (PRRS), bursal disease virus, caprine arthritis and encephalitis virus, and hepatitis virus, antigens (e.g., F1-V, PspA, rPA) from bacteria such as Mycobacterium tuberculosis, Clostridium tetani (e.g., tetanus), Mycobacterium avium paratuberculosis, Anaplasma, Borrelia, Leptospira, Ehrlichia, Brucella, Vibrio, and Aeromonas, antigens from fungal organism such as Cryptococcus, Histoplasma, Pneumocystis, and Aspergillus, antigens from protozoans such as Plasmodium (malaria), Leishmania, Babesia, Eimeria, and Icthyopthiris multifiliis, and antigens from nematodes such as Ascaris, Ancyclostoma, Necator, Oesophagostomum, and Haemonchus. Examples of immunogens of an anti-cancer vaccine that can be formulated into a vaccine depot and used as described herein include, without limitation, cancer antigens such as MUC-1, CA-125, Prostate serum antigen, and mesothelin. Examples of immunogens of an immunocontraception vaccine that can be formulated into a vaccine composition and used as described herein include, without limitation, antigens such as gonadotropin-releasing hormone, gonadotropin-releasing hormone linked to carrier proteins such as the mollusk hemocyanins blue protein, keyhole limpet hemocyanian, or ovalbumin, luteinizing hormone, luteinizing hormone beta subunit, and porcine zona pellucida. In some cases, a vaccine depot can include a multiple antigenic peptide of GnRH dimer as described elsewhere (Beekman et al., Vaccine, 17:2043-2050 (1999)). A vaccine composition can include any appropriate amount of an immunogen. For example, a vaccine depot can be formulated to include between about 500 ng and about 1 mg by weight of an immunogen of, for example, about 10,000 and 100,000 MW (e.g., about 18,000 MW).

The drug container may have any convenient configuration. In some instances, the drug container is configured as a syringe, such that it includes a tubular body having a plunger at a proximal end and an orifice at a distal end, e.g., for providing passage of the active agent composition from the inside of the container into a delivery structure, such as a needle, which may be operably engaged to the distal end of the container. The drug container may be fabricated from any convenient material, including glasses, plastics (such that the container is a polymeric container), etc. Suitable materials include, but are not limited to, those described in published PCT application publication nos. WO 2013/178771; WO2015/173260; WO2017/087798 and WO2017/085253; the disclosures of which are herein incorporated by reference.

In addition to a container, the therapeutic agent delivery system may also include tissue injector. The tissue injector is an element configured to convey the therapeutic agent composition from the container to, and in some instances into, a target tissue delivery site. In some instances the injector is a needle or cannula. The injector may have any convenient dimensions, and in some instances is has a gauge ranging from 20 to 35, such as 27 to 35, e.g., 30 to 33, such as 29, 29½, 30, 31, 32, 33 and 34 gauge. The injector may be made of any convenient material, e.g., stainless steel, etc.

The proximal end of the injector, e.g., needle, may be operably engaged with the distal end of the active agent container, using any convenient configuration. Examples of suitable configures include, but are not limited to, press fit configurations, luer fitting configurations, etc.

Therapeutic agent delivery systems of the devices further include a tissue contacting tip, where the tissue contacting tip may be stably associated with the injector, e.g., needle. The tissue contacting tip may be configured to prevent contact with a tissue structure near a target tissue delivery site, e.g., an ocular lens or ocular retina where the target tissue delivery site is an ocular tissue delivery site. For example, the tissue contacting tip may be configured to extend only a certain limiting distance beyond the target tissue contacting end of the body of the tissue delivery component. While this limiting distance may vary, in some instances the limiting distance ranges from 0.5 to 8 mm, such as 3 to 4 mm. The tissue contacting tip may be a structure having a proximal end attached to the needle and a distal end extending beyond the distal end of the needle by a distance ranging from 2 to 20 mm. In some instances, a locking mechanism maintains the distal end of the needle relative to the distal end of the tissue contacting tip prior to actuation. The locking mechanism may be releasable upon movement of the tissue contacting tip relative to the needle, such as a rotational movement, e.g., of the tissue contacting tip about the central longitudinal of the needle. For example, a tissue contacting tip may be turned, such as a quarter turn, relative to the associated needle which results removing the lock and allowing the needle to be moved relative to the distal end of the tissue contacting tip. Where the therapeutic agent delivery system includes an injector, such as a needle or cannula, the distal, tissue contacting end of the tissue contacting tip may include an opening dimensioned to provide for passage of the distal end of the injector through the contacting tip during therapeutic agent delivery, thereby providing access of the needle directly to a target tissue. Upon actuation, the distal end of the needle moves relative to the distal end of the tissue contacting tip. The distance that the distal end of the needle extends beyond the distal end of the tissue contacting tip during actuation and active agent delivery may vary, and in some instances ranges from 0.1 to 5 mm, such as 0.5 to 4.0 mm. While the dimensions of the opening, when present, may vary, in some instances the opening has a diameter sufficient to accommodate passage of a needle having a gauge, e.g., as described above. The distal end of the tissue contacting tip may in some instances be fabricated from a thermally conductive material, e.g., a metal or alloy thereof, such as in those instances where the distal end of the tissue contacting tip operatively engages the distal end of a cooling element of a cooling system of the actuator component, e.g., as described in greater detail below. In some instances, the tip may be configured to ensure proper, operable, engagement of the therapeutic agent delivery system with the actuator. For example, the tip may be configured to engage in a specific orientation with the cold arm of the pain mitigation component of the actuator, e.g., as described in greater detail below, in only one way, e.g., by having a flat top and a groove or other alignment component that engages with the cold arm.

In some instances, the therapeutic agent delivery system may include one or more filters. The filters may be configured to remove particles or other unwanted components present in the therapeutic agent composition prior to delivery to the target tissue delivery site. Such filters may be configured to inhibit passage of particles above a certain pore size from >0.1 μm to >50 μm, such as >5 μm. The one or more filters may be positioned at any convenient location in the therapeutic agent delivery system, e.g., at the exit from the container into the injector, at some point along the injector, at the distal end of the injector, etc.

In some instances, the dimension of the tissue contacting tip will be such that injector, e.g., needle, entry occurs at a predetermined distance from the corneal limbus (where distances may be as provided above) when the tissue contacting tip is placed on the ocular surface, where such configurations may negate any need for manual measurement for safe injection distance prior to procedure (intravitreal, intracameral etc.).

Where desired, the distal, tissue contacting end of the therapeutic agent delivery component may include a removable cover, e.g., that is present until the device is used to deliver therapeutic agent to a target delivery site. The cover may be configured as a release liner or analogous structure, such that it may be easily removed just prior to use. The cover may be sterile or sanitized as desired, and fabricated from any convenient material, e.g., plastics, etc. It may also take the form of a sterile peel pack, sterile box, etc.

In some instances, the tip may include a mechanism configured sequester liquid active agent composition expelled during a priming step, e.g., so that such composition does not contact the surface of the target ocular location. An example of such a sequestration mechanism may be an absorbent member configured to soak up any such liquid, where in some instances the absorbent member may be configured to transport such liquid, e.g., via a wicking action, away from the tissue contacting surface of the tip. In some instances, excess fluid can be removed via active suction in the tip. In some instances, excess fluid can be removed via a siphon mechanism.

The therapeutic agent delivery system may, where desired, include an antimicrobial element. The antimicrobial element may be any convenient element having antimicrobial properties and be positioned at one or more locations of the therapeutic agent delivery system. For example, the antimicrobial element may be positioned at the distal, tissue contacting end of the body in order to provide for at least aseptic conditions during contact of the device to the target tissue delivery site, in order to sanitize the target tissue delivery site, etc. The antimicrobial element may include an antimicrobial agent, which may be present in a holder, such as a matrix material, reservoir, etc. As with the therapeutic agent, the antimicrobial agent, when present, may be present in a composition that is in a variety of different physical steps, including liquid, solid, semi-solid, and gaseous. Antimicrobial agents of interest include, but are not limited to: povidone-iodide (Betadine), chlorhexidine (Nolvasan), ethanol or other alcohols, and the like.

The therapeutic agent delivery system may, where desired, include an analgesic/anesthetic agent. When present, the analgesic/anesthetic agent may be present in any convenient manner that provides for delivery of the analgesic/anesthetic agent to the target tissue delivery site during use of the device. For example, the analgesic/anesthetic agent may be positioned at the distal, tissue contacting end of the tip in order to provide for at least aseptic conditions during contact of the device to the target tissue delivery site. The analgesic/anesthetic agent may be present in a holder, such as a matrix material, reservoir, etc. As with the therapeutic agent, the analgesic/anesthetic agent, when present, may be present in a composition that is in a variety of different physical steps, including liquid, solid, semi-solid, and gaseous. Analgesic/anesthetic agents of interest include, but are not limited to: lidocaine, benzocaine, prilocalne, lidocaine, dubicaine, mepivacaine, bupivacaine, and the like; naturally-derived products, such as saxitoxin, neosaxitoxin, tetrodotoxin, menthol, eugenol, and cocaine, and the like; etc.

As indicated above, in some instances, the therapeutic agent delivery system may include a component of a locking element for release-ably engaging the therapeutic agent delivery system in a receiving space of the actuator component of the device. Any convenient locking mechanism may be employed, such as but not limited to: press fit (e.g., in the form of ridges and corresponding notches, a clip in the receiving space configured to press fit engage), mechanical, e.g., a movable arm that moves between a first position that holds the delivery system in the delivery space and a second position that does not, and the like. The locking element component of the locking element that is present on the therapeutic agent delivery component may vary, as desired, and is selected based on the companion element that is present on the actuator, where examples of such locking components include one or more ridges for press fitting into corresponding notches, one or more structures that press fit into a clip, and the like.

In some instances, the therapeutic agent delivery system further includes one or more identifiers. In some instances, an identifier present on the therapeutic agent delivery system is an identifier that is configured to be read by an identifier reader of the actuator component of the device. While such reader compatible identifiers may vary, in some instances the identifier is a barcode, such as a linear barcode or a matrix barcode, such as a QR code. In some instances, the reader compatible identifier is a radio frequency identification (RFID) tag, such as a near field communication (NFC) tag, where the RFID tag may be passive or active. Information included in the identifier may include, but is not limited to, identity of the therapeutic agent (brand name and/or generic name), date of manufacture, date of expiry, source of manufacture, dosage amount, drug concentration, intended route of administration, handling and storage information, delivery volume, indication for use, lot number, etc.

In addition to, or instead of, a reader compatible identifier, the therapeutic agent delivery system may include an identifier that is visual identifier, such that it is configured to be read by a health care practitioner. Visual identifiers are identifiers that may be readily understood by a human upon looking at the identifier, such that computer processing of the identifier is not required. Examples of such identifiers include, but are not limited to, text identifiers, color coding identifiers, commonly understood symbols, identifying trademarks, logos, and the like. Information conveyed by the visual identifier may vary as desired, where examples of information that may be conveyed by the visual identifier include, but are not limited to: information about the therapeutic agent delivery component or therapeutic agent present therein, such as identity of the therapeutic agent (brand name and/or generic name), date of manufacture, date of expiry, source of manufacture, dosage amount, drug concentration, intended route of administration, handling and storage information, delivery volume, indication for use, lot number, etc.

The entire therapeutic agent delivery system may be configured for single use, such that the entire therapeutic agent delivery system is disposable. Alternatively, one or more components of the therapeutic agent delivery system may be reusable. For example, the container of the therapeutic agent delivery component may be reusable, e.g., where the container may be sterilized, such that the component may be used multiple times, e.g., by reloading therapeutic agent into the therapeutic agent delivery system.

The therapeutic agent delivery system components of the invention may be fabricated using any convenient materials or combination thereof, including but not limited to: metallic materials such as tungsten, copper, stainless steel alloys, platinum or its alloys, titanium or its alloys, molybdenum or its alloys, and nickel or its alloys, etc.; polymeric materials, such as polytetrafluoroethylene, polyimide, PEEK, and the like; ceramics, such as alumina (e.g., STEATITE™ alumina, MAECOR™ alumina), etc. The drug reservoir can be made of plastic, such as polypropylene or polystyrene, or any material commonly used for syringes and the like. It can also be made of glass, including type 1 glass, as is commonly used for long-term storage of drugs and biologics. Alternatively, it can be made of non-leachable plastic materials that are used for long-term storage of drugs or biologics, such as cyclic olefin copolymer (Crystal Zenith) and the like.

### Actuator Component

Also present in the devices of the invention is an actuator component. As described above, the actuator component is configured to operably engage with a therapeutic agent delivery system such as described above, to produce a therapeutic agent delivery device of the invention. Aspects of actuator components according to embodiments of the invention include a body having a proximal end and a distal end, a therapeutic agent delivery system receiving space configured to be operably, and in some instances release-ably, engaged with a therapeutic agent delivery system, e.g., as described above. Further aspects of the actuator component include a therapeutic agent delivery system actuator made up of one or more subcomponents and configured to actuate a therapeutic agent delivery system. The actuator further includes one or more components of a pain mitigation system configured to mitigate pain in target tissue delivery site, and in some instances the actuator may include all of the components of a pain mitigation system.

The therapeutic agent delivery system actuator is an element or subsystem that is configured to actuate the therapeutic agent delivery system so as to deliver a therapeutic agent to a target tissue delivery site. The nature of the therapeutic agent delivery system actuator may vary, e.g., depending on the nature of the therapeutic agent delivery system. For example, where the therapeutic agent delivery system includes a container, e.g., a syringe, operably engaged to a tissue injector, e.g., a needle, the therapeutic agent delivery system actuator may be configured to provide for control of one or more of angular position, linear position, velocity and acceleration of the tissue injector. In some instances, the actuator, either alone or in conjunction with a guiding element of the therapeutic agent delivery component, is configured to provide for an angle of the injector relative the distal, tissue contacting end of the therapeutic agent delivery component, that ranges from 0 to 90, such as 75 to 90°. In some embodiments, the therapeutic agent delivery system is present in the device at a pre-determined angle, for example, 90 degrees to the biologic tissue when the cold tip is applied to the biologic tissue, so that when the device tip is placed on the eye abutting the limbus and causing very slight indentation of the ocular surface 360 degrees around the tip, the needle tip will reproducibly be inserted into the eye at a defined, safe angle posterior to the limbus of the eye to avoid the danger of striking the retina, zonules, or lens. In some instances, the actuator provides for a velocity of introduction of the injector into a target tissue delivery site that ranges from 1 to 100 mm/sec, such as 1 to 10 mm/sec, including 3.5 to 7 mm/sec. The therapeutic agent delivery system actuator may be configured to provide for control of release of a therapeutic agent from the therapeutic agent container. The therapeutic agent delivery system actuator may be configured to provide for controlled removal of the tissue injector from the target tissue delivery site. In some such instances, the actuator may be configured to withdraw the injector from a target tissue delivery site at a velocity ranging from 1 to 10 mm/sec, such as 3.5 to 7 mm/sec. In some instances, the therapeutic agent delivery system actuator is configured to prime the tissue injector, e.g., where the therapeutic agent delivery system includes an amount of gas, e.g., air (such as in the form of bubbles) and the actuator removes the gas from the system, e.g., by causing the gas to evacuate from the system via the injector. In some instances, the actuator is configured to sequentially move the drug container in a first priming motion and a second injection motion. In some instances, the actuator is further configured to withdraw the needle back into the device following injection of the active agent composition

The therapeutic delivery system actuator may vary as desired. Examples of therapeutic delivery system actuators that may be employed in embodiments of the invention and present in the actuator component include, but are not limited to: motorized actuators (including those that include a micro-motor, such as a stepper motor, direct current (DC) motor, brushless motor and the like), as well as non-motorized actuators, e.g., pneumatic powered actuators, hydraulically power actuators, spring-loaded actuators, manually operated actuators, e.g., plunger comprising actuators, and the like. The functionality of the therapeutic agent delivery system actuator may be controlled by one or more modules, as desired. Motorized actuators may be coupled to gearheads, spindle drives and the like. An encoder may be used to provide precise motor control.

As mentioned above, the actuator component may include one or more subcomponents that work to achieve the desired actuation, e.g., plunger depression and active agent delivery, such as described above. For example, wherein the actuator component is a motorized actuator component, the actuator component may include one or more motors, such that in some instances the actuator component includes a plurality of motors, e.g., where each member of the plurality of motors provides for a distinct unidirectional movement. For example, a motorized actuator having a plurality of motors may be configured such that a first motor is used in a priming step, e.g., as described to above, to move the needle into the eye, and depress the plunger, while a second motor of the plurality may be employed to pull the needle/syringe back into the device. Such configurations can avoid using a complex track and may be a simpler overall design. Alternatively, a given actuator may be a hybrid actuator that includes two or more different types of actuators, such as a motor to performing the priming, needle movement and plunger depression, and a spring to push the needle/syringe back into the device.

In addition to the therapeutic agent delivery system actuator, the actuator component may include one or more components of, including all of, a pain mitigation system, e.g., as described above. Specific cooling systems of interest that may find in anesthesia producing pain mitigation systems may vary, where cooling systems of interest include, but are not limited to: thermoelectric cooling systems, liquid evaporation cooing systems, Joule-Thompson cooling systems, thermodynamic cycle cooling systems, endothermic reaction cooling systems and low-temperature substance cooling systems. The pain mitigation system may or may not include a tissue engager, such as a cool tip, e.g., depending on whether the tissue engager is integrated with the therapeutic agent delivery component, e.g., as described above.

In some instances, the pain mitigation anesthesia producing system is a thermoelectric cooling system, e.g., one that includes one or a combination of thermoelectric (Peltier) devices. While thermoelectric cooling systems employed in embodiments of devices of the invention may vary, in some instances the thermoelectric cooling systems include a cold tip that is configured to contact a target tissue delivery site (and therefore may also be referred to as a tissue engager), a power source, a controller, a cooling power concentrator, one or more Peltier unit modules, and a heat sink. It should be understood that, in some embodiments, a given thermoelectric cooling system may include a heating element (not shown) that operates in conjunction with the cooling elements to precisely maintain a desired temperature and/or heat flux. It should be understood that, in various embodiments, these elements may reside in the actuator component, the therapeutic agent delivery component, or, in some embodiments, elements reside in both the actuator component and the therapeutic agent delivery component.

In some embodiments, the cold tip (i.e., tissue engager) is made of a thermally conductive material (i.e., it includes a thermally conductive member), such as a metal (where metals of interest include, but are not limited to copper, gold, zinc, aluminum and the like), and can be sized to be generally equal to or smaller than the target tissue delivery site (e.g., the area of the ocular or other biologic surface to which the therapeutic agent is to be delivered). In some embodiments, the end of the tissue engager is circular, having a diameter ranging from 1 to 10 mm, such as 2 to 8 mm, or about 5 mm. Where desired, a thermally insulating outer ring member (e.g., that corresponds to the target area to be cooled) may be included. When present, the thermally insulating outer ring member restricts the area being cooled within the target area, which is touched by the thermally conductive cold tip, preventing damage to adjacent cells outside the target area. The cold tip may have any convenient shape, including but not limited to cylindrical, polygonal, oval, crescent, or any other conducive shape. It is noted that the tissue engager of the pain mitigation system may be integral with the other parts of the cooling system, or may be detachable from the other parts of the cooling system of the actuator, such that it is release-ably engaged to the actuator and pain mitigation system thereof, where release-ably engaged is as describe above.

In some embodiments, the power source of the pain mitigation system includes a portable power source, such as a battery, capacitor, or similar device. In some embodiments, the power source includes a rechargeable lithium ion battery pack (e.g., 28 Wh), which provides sufficient energy on a single charge to operate the device for a sufficient period of time, e.g., 0.5 to 2.5 hours, such as 1 hour. In some embodiments, the power source can include a non-portable power source. In some embodiments, the power source can be a non-rechargeable battery. In some embodiments, the device is configured to allow easy battery replacement.

The controller may include a temperature regulating feedback loop to maintain highly accurate temperature control and/or a timed lockout mechanism to prevent excessive cooling. In some embodiments, the controller can include a temperature sensor operably coupled with at least one member of a thermal circuit comprising the cold tip, a cooling power concentrator, one or more Peltier unit modules, a heat sink, the surrounding environment, and the target tissue delivery site to output a temperature signal in response to a detected temperature. In this way, the controller receives the temperature signal and is operable to control an operating temperature of Peltier unit module(s) via controlled current flow, controlled voltage, and/or pulse width modulation (PWM) of a power source, e.g., a DC battery source, thereby precisely regulating an operating temperature of thermoelectric cooling system. In some embodiments, the temperature sensor is arranged to directly measure the temperature of the target tissue delivery site or any portion of the thermal circuit using any one or a number of thermal sensors, such as but not limited to thermistors, thermocouples, and resistance or tissue thermometers. The controller can then compute temperature and/or heat flux. In some instances, the controller is configured to maintain a predetermined temperature or temperature range using a constant value, a pulse of certain magnitude and duration, or a more complex prescribed pattern. In some embodiments, the controller is configured to automatically power off if the tissue engager temperature falls below a certain temperature (e.g., −40° C., −35° C., −30° C., −25° C., −20° C., −15° C., −10° C., −5° C.) to ensure a safe operating temperature range, and/or if a battery temperature exceeds a certain temperature, e.g., 70° C. or the heat sink temperature exceeds a certain temperature, e.g., 140° C. In some embodiments, controller can operate on the basis of applied, measured, or desired heat fluxes rather than applied, measured, or desired temperatures.

Thermoelectric cooling systems finding use in embodiments of the invention may include a cooling power concentrator. In some embodiments, the cooling power concentrator may include an elongated concentrator made of a thermally-conductive material, such as but not limited to metal, e.g., as described above. The cooling power concentrator can be disposed along a central longitudinal axis of the actuator, and may collect cooling power from one or multiple Peltier units. In some embodiments, the cooling power concentrator can be polyhedron in shape, and the cooling power collected from the surface(s) in contact with Peltier unit(s) is concentrated to one or more surfaces whose aggregate area is less than that of the Peltier unit cooling surface(s) at which collection occurs. However, it should be understood that the cooling power concentrator can have other shapes, including cylinder, cone, conical cylinder, sphere, hemisphere, or any other shapes that provide collecting and concentrating of cooling power. In such embodiments, the Peltier unit module(s) can be shaped to define a complementary surface to enhance surface area contact between Peltier unit module(s) and the cooling power concentrator to facilitate thermoelectric cooling.

In addition to the above component, a thermoelectric cooling system may include a heat sink. In some instances, a heat sink is made of a thermally conductive material to efficiently spread the heat rejected from Peltier unit module(s) of the system. In some embodiments, a heat sink is radially disposed about cooling power concentrator and Peltier unit module(s). In other words, a heat sink radiates outwardly from a central longitudinal axis of the actuator component. However, it should be understood that a heat sink can radiate heat in other directions depending on the relative angle of the hot surface of Peltier unit module(s) with respect to central cooling portion of cold tip.

Further details regarding embodiments of thermoelectric cooling systems that may be employed in devices of the invention are provided in U.S. Published Patent Application Publication No. 20160279350; the disclosure of which is herein incorporated by reference.

As indicated above, in some instances, the actuator component may include a component of a locking element for release-ably engaging the therapeutic agent delivery system in the receiving space of the actuator component of the device. As described above, any convenient locking mechanism may be employed, such as but not limited to: press fit (e.g., in the form of ridges and corresponding notches, a clip in the receiving space configured to press fit engage), mechanical, e.g., a movable arm that moves between a first position that holds the delivery system in the delivery space and a second position that does not, and the like. The locking element component of the locking element that is present on the actuator component may vary, as desired, and is selected based on the companion element that is present on the actuator, where examples include a notch configured to press-fit engage a ridge on the delivery device, a clip configured to receive a component of the delivery device, etc.

In some instances, the actuator component includes an identifier reader for reading an identifier of a therapeutic agent delivery component release-ably engaged with the actuator component. The identifier reader may vary, as desired, depending on the nature of the identifier that is associated with the therapeutic agent delivery component. For example, where the identifier is a barcode, the identifier reader of the actuator may be any convenient barcode or QR code scanner. Likewise, where the identifier is a radiofrequency identifier, the identifier reader of the actuator may be any convenient RFID reader. The identifier reader, when present, is located on the actuator at a position such that it is reading relationship with the identifier of a therapeutic agent delivery component when release-ably engaged with the actuator.

Where the actuator component includes an identifier reader, in some instances the actuator is configured to be active only when the identifier reader detects an acceptable identifier. An acceptable identifier may be an identifier that imparts one or more types of information upon which acceptability may be based, such as but not limited to: whether the therapeutic agent delivery component has is filled with the correct therapeutic agent, whether the therapeutic agent delivery component is expired, where the therapeutic agent delivery component is manufactured by an acceptable, authentic source; whether the therapeutic agent delivery component has been previously registered as lost, etc. In such instances, the reader may be coupled to an actuator control element that only enables one or more actuator components, such as the therapeutic agent delivery system actuator, the pain mitigation system, etc., when an acceptable identifier is read by the identifier reader. As such, where an unacceptable identifier is read by the reader, the reader may send a single to the controller that disables one or more of the actuator components. Alternatively, where an unacceptable identifier is read by the reader, the reader may send a single to the controller that one or more of the actuator components should not be enabled.

Actuator components of the invention may further include a communications module, which module is operably coupled to one or more components of the actuator and provide for data transfer therefrom to another component, e.g., an external device, etc. The communications module may be configured to provide for the transfer of data in a wired or wireless mode, as desired. For example, the communications module may be configured to wirelessly transfer data, e.g., with a networked device, while be used, and then transfer data using a wired configuration when docked at a docking station, such as described below. Communications modules of the actuators may be configured, e.g., via hardware and/or software implementation, to perform desired communications functions, e.g., to receive data from an actuator element, to transfer data, e.g., to a USB port for wired communications or a wireless transmitter for wireless communications, etc. Communications modules (as well as any other modules described herein, such as actuator controller modules, etc.) are made up of one or more functional blocks which act in concert to perform a particular function, which is the purpose of the module. A given communications module may be implemented as hardware, software or a combination thereof. In some instances, the communications module may include a circuitry element, such as an integrated circuit. When present, integrated circuits may include a number of distinct functional blocks, i.e., modules, where the functional blocks are all present in a single integrated circuit on an intraluminal-sized support. By single integrated circuit is meant a single circuit structure that includes all of the different functional blocks. As such, the integrated circuit is a monolithic integrated circuit (also known as IC, microcircuit, microchip, silicon chip, computer chip or chip) that is a miniaturized electronic circuit (which may include semiconductor devices, as well as passive components) that has been manufactured in the surface of a thin substrate of semiconductor material.

Where desired, actuator components may include a variety of different types of power sources that provide operating power to the actuator component in some manner. The nature of the power source may vary, and may or may not include power management circuitry. In some instances, the power source may include a battery. When present, the battery may be a onetime use battery or a rechargeable battery. For rechargeable batteries, the battery may be recharged using any convenient protocol. In some applications, the actuator may have a battery life ranging from 0.1 to 14 hrs, such as 0.5 to 10 hrs or 1 hour to 5 hours.

In certain instances, the actuator of the invention includes an updatable control module, by which is meant that the actuator is configured so that one or more control algorithms of the actuator may be updated. Updating may be achieved using any convenient protocol, such as transmitting updated algorithm data to the control module using a wire connection (e.g., via a USB port on the device) or a wireless communication protocol. The content of the update may vary. In some instances, a actuator component is updated to configure the unit to be used with a particular therapeutic agent delivery component. In this fashion, the same actuator component may be employed with two or more different therapeutic agent delivery components that may differ by from each other in one more ways, e.g., identify of therapeutic agent, manufacturer of therapeutic agent delivery component, etc. The update information may also include general functional updates, such that the actuator component can be updated at any desired time to include one or more additional software features and/or modify one or more existing programs of the device. The update information can be provided from any source, e.g., a particular elongated member, the internet, etc.

The actuator component may include one or more safety mechanisms, e.g., in addition to or instead of, the identifier/reader compatibility mechanism as described above. In some embodiments, the therapeutic agent delivery system actuator will provide for actuation only if a switch is depressed continuously during the injection process. In some embodiments, there will be a safety mechanism to halt injection. For example, the actuator component may include a limit switch configured to modulate the activity of the actuator. For example, the actuator component may include a limit switch (e.g., an optical or mechanical limit switch) configured to modulate activity of a motor, e.g., to prevent movement of a plunger by the motor beyond a defined distance. Such a limit switch may be configured to prevent a malfunctioning motor from delivering too great a volume of fluid. In such instances, a limit switch can be set to any volume, such as a volume ranging from 10 to 50 μl, from 25 to 75 μl, from 75-125 μl, and from 75 to 3000 μl. Another example of a safety mechanism is a mechanism configured to prevent use of the actuator by a non-authorized user. For example, the actuator may be configured to only be activated upon recognition of an authorized user, e.g., by input of an authorization code, fingerprint identification, facial recognition, etc. When so configured, any convenient user identification recognition hardware/software may be employed.

The actuator components of the invention may be fabricated using any convenient materials or combination thereof, including but not limited to: metallic materials such as tungsten, stainless steel alloys, platinum or its alloys, titanium or its alloys, molybdenum or its alloys, and nickel or its alloys, etc.; polymeric materials, such as polytetrafluoroethylene, polyimide, PEEK, and the like; ceramics, such as alumina (e.g., STEATITE™ alumina, MAECOR™ alumina), etc.

In some instances, the actuator component may include a display. By display is meant a visual display unit, which may include a screen that displays visual data in the form of images, lights, and/or text to a user. The screen may vary, where a screen type of interest is an LCD screen. The display, when present, may be integrated with the actuator component. As such, the display may be an integrated structure with the actuator component, such that it cannot be separated from the actuator component without damaging the monitor in some manner. The display, when present will have dimensions sufficient for use with the actuator, where screen sizes of interest may include 100 cm² or smaller, such as 20 cm² or smaller, e.g., 10 cm² or smaller, 5 cm² or smaller, including 2 cm² or smaller, etc., where the screen will have dimensions sufficient to display the desired information to a user, e.g., 0.5 cm₂ or larger, such as 1 cm² or larger. The display may be configured to display a variety of different types of information to a user, where such information may include device settings (such as tip temperature, time of cooling application (e.g., numerical or visual, such as a decreasing bar graph) etc.), therapeutic agent information (e.g., drug name or identification, dose, therapeutic agent expiration date, manufacturing and/or handling data (such as manufacturer, lot number, manufacture date, shipping data, etc.), device status (such as battery life indicator, connectivity indicator (e.g., WiFi connectivity, cellular signal, etc.), and the like.

### Specific Embodiments

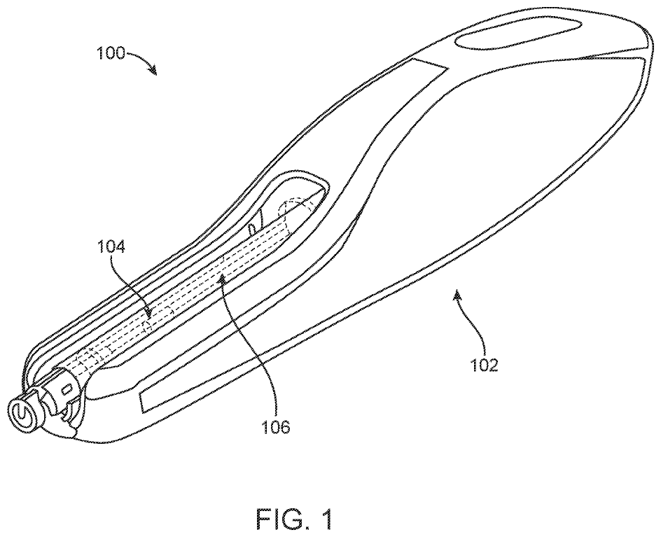

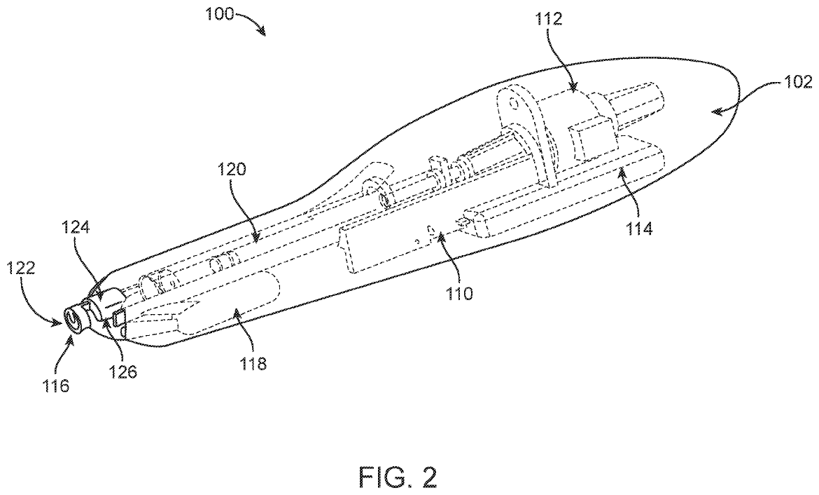

FIG. 1 provides a view of a hand held therapeutic agent delivery device 100 according to an embodiment of the invention. As shown in FIG. 1, the device 100 includes an actuator component 102 and a therapeutic agent delivery system 104 operably engaged in a receiving space 106 of the actuator component. FIG. 2 provides a cutaway view of the device shown in FIG. 1. As shown in FIG. 2, the actuator component 102 includes a body that houses an actuator subsystem, i.e., an auto injector mechanism 110, a stepper motor 112 (such as 19000 series Captive Haydon G4 Stepper Motor), a battery 114 and a pain mitigation cooling system that includes a cooling element 116 and a cooling module 118. The therapeutic agent delivery system 104 includes a syringe and needle 120 as well as a tissue contacting tip 122 having an RFID tag 124 which are present in the receiving space, where the distal end of the tissue contacting tip 122 is operably engaged with the distal end of the cooling element 116. Also shown is RFID reader 126 which is part of the actuator component 102.

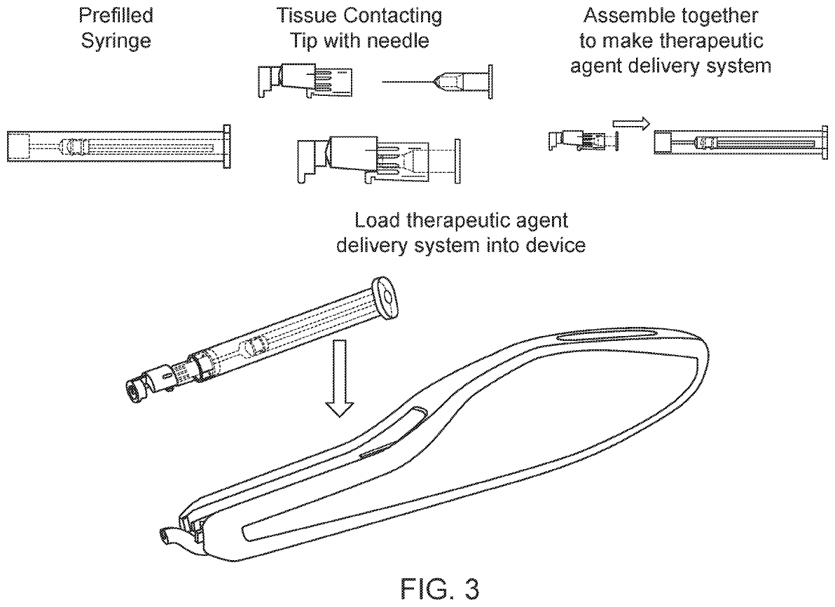

FIG. 3 provides various views of a therapeutic agent delivery system and components thereof, according to an embodiment of the invention. As shown in FIG. 3, the therapeutic agent delivery system includes a drug container in the form of a prefilled syringe operably coupled to a composition structure made up of a delivery needle pink and a tissue contacting tip (light blue/grey) associated therewith. In the configuration illustrated in FIG. 3, the proximal end of the tissue contacting tip (grey) is associated with the needle (pink). The distal end of the tissue contacting tip (light blue) is maintained at a defined distance from the distal end of the needle by a locking mechanism operably joining the proximal (grey) end to the distal (light blue) end. As such, the locking mechanism is made up of a ridge and groove. Prior assembly of the therapeutic agent delivery system, the ridge is not aligned with the groove and therefore the distance between the proximal end of the tissue contacting tip and the distal end of the needle is maintained. Following assembly and prior to placing in the receiving space of the actuator, the distal end of the tissue contacting tip is turned such that the ridge aligns with the groove of the proximal end of the tissue contacting tip, thereby removing the lock and the allowing moving of the distal end of the needle relative to the proximal end of the tissue contacting tip.

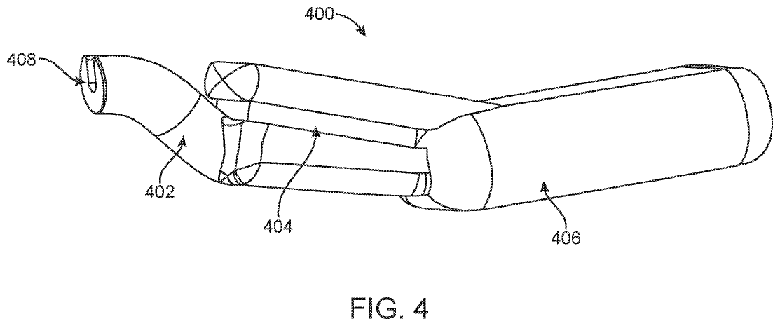

FIG. 4 provides a view of a cooling system of the device shown in FIGS. 1 and 2. As shown in FIG. 4, the cooling system 400 of the actuator component includes a metal tip or cooling arm 402 coupled to a thermoelectric (TEC) module 404. Coupled to the metal tip 402 and TEC 404 is a chilled heat sink 406. The heat sink may be a solid material, such as a metal. Alternatively, the heat sink may include a liquid medium. As shown, the distal end of metal tip 402 is configured to operably engage with the distal end of a tissue contacting tip and includes a groove 408 that allows for passage of the distal end of a needle upon actuation of the device.

In using the device of FIGS. 1 to 4, the autoinjector is driven by a small stepper motor—no springs are required. The pre-filled syringe and tissue contacting tip are inserted into the device prior to use. The completion of cooling is indicated by a timer and an audible alarm. Once cooling is complete, the autoinjector priming is started by the physician. Injection is initiated after priming is complete. Depth of needle penetration is 3.5 mm+/−1 mm. In various embodiments, needle penetration may have a depth ranging from 0.5 to 10 mm, such as 1.0 to 5.0 mm. Total drug delivery time is 3-5 seconds.

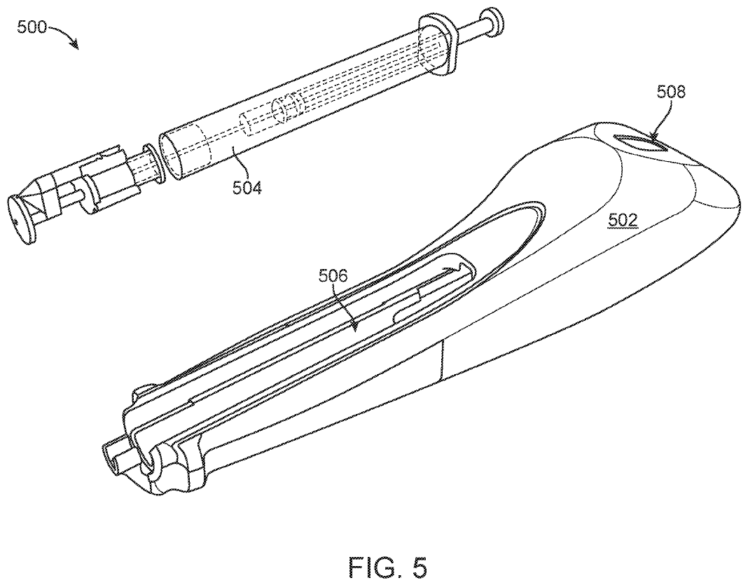

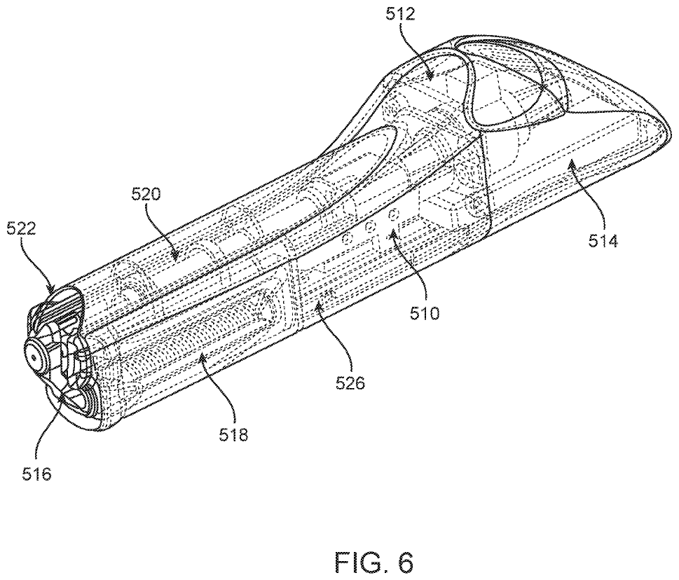

FIG. 5 provides a view of a device according to another embodiment of the invention. As illustrated in FIG. 5, device 500 includes an actuator component 502 and a therapeutic agent delivery system 504 which can be operably engaged in a receiving space 506 of the actuator component. Also shown is display 508. FIG. 6 provides a cutaway view of the device shown in FIG. 5. As shown in FIG. 6, the actuator component 502 includes a body that houses an actuator subsystem, which subsystem includes an auto injector mechanism 510, a stepper motor 512, a battery 514 and a pain mitigation cooling system that includes a cold arm 516 and a cooling module 518. The therapeutic agent delivery system 504 includes a syringe 520 as well as a single use, sterile tissue contacting tip 522, where the distal end of the tissue contacting tip 522 is operably engaged with the distal end of the cold arm 516. Also shown is RFID reader 526 which is part of the actuator component 502 and reads an RFID tag of the tip 522.

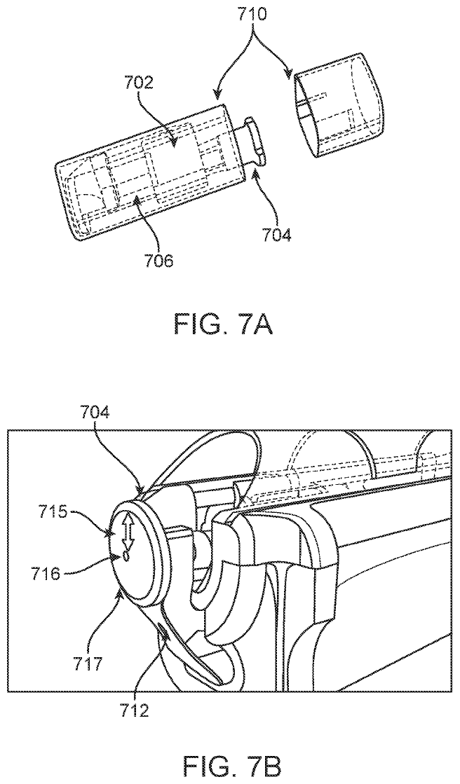

FIG. 7A provides a view of a composite sterile tip/needle assembly in a sterile packaging that may be employed with the device illustrated in FIGS. 5 and 6. In FIG. 7A, the sterile tip composite 702 includes a 32-gauge needle 704 present in a needle sheath 706. The composite is present in packaging 710. FIG. 7B shows the sterile tip during use, when operably engaged with the cold arm 712 of the actuator component. Sterile tip 714 has a 3.5 mm diameter distal end 715 and includes a central hole 716 dimensioned to allow passage of a 32-gauge needle therethrough. Covering the distal end of the sterile tip is metal foil 717.

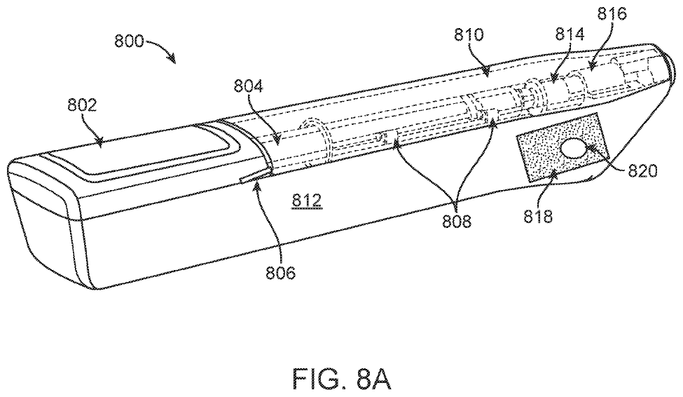

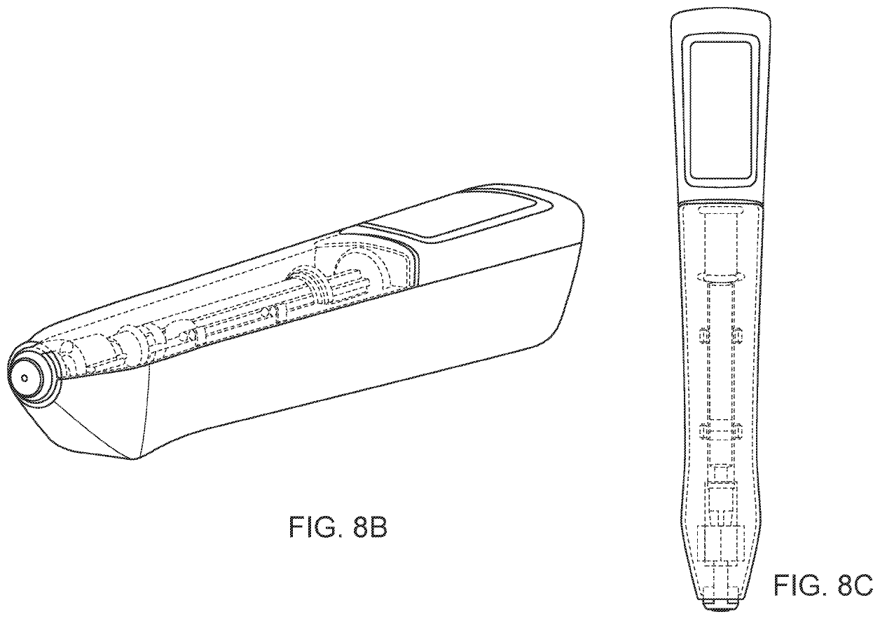

FIGS. 8A to 8C provide differing views of a hand held therapeutic agent delivery device 800 according to an embodiment of invention. As shown in FIG. 8A, the device 800 includes an actuator component 812 having a therapeutic agent delivery system operably engaged in a receiving space thereof. The actuator component 8122 includes a body that houses an actuator subsystem, i.e., an auto injector mechanism, a stepper motor, a battery and a pain mitigation cooling system that includes a cooling element and a cooling module. The therapeutic agent delivery system includes a syringe and needle as well as a tissue contacting tip which are present in the receiving space, where the distal end of the tissue contacting tip is operably engaged with the distal end of the cooling element. As illustrated in FIG. 8A, the actuator component includes cover 810 on a hinge 806 and a display 802. The cover 810 is transparent, provide a view of the needle plunger 804, syringe clips 808, needle hub 814 and sterile tip 816. Also shown is a textured gripping portion 818 having an activation button 820. FIGS. 8B and 8C provide alternative views of the device shown in FIG. 8A.

### Docking Stations and Systems Including the Same

Aspects of the invention include docking stations that are configured to dock an actuator component, and systems that include a docking station and an actuator component. A docking station is a base unit or analogous device that is configured to engage with an actuator component, e.g., as described above. When engaged with an actuator component, the docking station may perform one or more functionalities, which functionalities may include, but are not limited to: maintaining a pain mitigation system in a desirable state (for example maintain a cooling system at a desired temperature); transferring data between the actuator component and an external device; sanitizing the distal end of an actuator component; recharging a power source of an actuator component; communicating with a computer, server, or database, and the like. The docking station may include a single actuator component dock, (i.e., a site or location configured to engagingly receive an actuator), or two or more actuator component docks, such that the number of actuator docks in a docking station may, in some instances, range from 1 to 6, such as 2 to 4. Docking stations of the invention may have any convenient configuration. Docking stations may be configured as table top devices, wall mounted devices, floor devices, etc., as desired.

In order to provide different desired functionalities, the docking station may include a number of a different subsystems or components. For example, a docking station may include a cooling system, e.g., that is configured to maintain the temperature of docked actuator and/or therapeutic agent delivery components in a desired range. Examples of suitable cooling systems include, but are not limited, those described above. The docking station may include a communications module, e.g., for mediating data transfer between docked actuator and/or therapeutic agent delivery components and a module of the docking stations and/or an external device. The docking station may include a power module, e.g., for recharging a power source of a docked actuator. The docking station may include detector, e.g., for detecting docked actuator and/or therapeutic agent delivery components. The docking station may include an identifier reader, e.g., for reading an identifier on a docked actuator and/or therapeutic agent delivery components, such as an identifier reader as described above. Where desired, the docking station may include an identifier, such as described above.

In some instances, the docking station may include a display. By display is meant a visual display unit, which may include a screen that displays visual data in the form of images, lights, and/or text to a user. The screen may vary, where a screen type of interest is an LCD screen. The display, when present, may be integrated with the docking station. As such, the display may be an integrated structure with the docking station, such that it cannot be separated from the docking station without damaging the display in some manner. The display, when present will have dimensions sufficient for use with the docking station, where screen sizes of interest may include 100 cm² or smaller, such as 20 cm² or smaller, e.g., 10 cm² or smaller, 5 cm² or smaller, including 2 cm² or smaller, etc., where the screen will have dimensions sufficient to display the desired information to a user, e.g., 0.5 cm₂ or larger, such as 1 cm² or larger. The display may be configured to display a variety of different types of information to a user, where such information may include docking station settings (such as internal temperature (e.g., as provided real time by thermocouples/thermistors), actuator information (e.g., actuator identification, manufacturing and/or handling data (such as manufacturer, lot number, manufacture date, shipping data, etc.), docking station status (such as battery life indicator, connectivity indicator (e.g., wifi connectivity, cellular signal, etc.), and the like. The display may include a graphical user interphase, enabling users to interact with the screen and modify specific dock settings (e.g., set the cooling temperature colder or warmer, etc.)

In addition to docking stations, e.g., as described above, aspects of the invention further include docking systems. Docking systems include a docking station having one or more actuator components docked therewith.

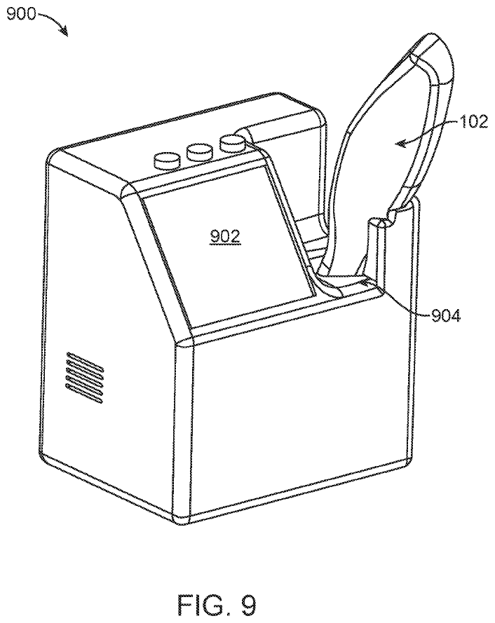

FIG. 9 provides a view docking station according to an embodiment of the invention. As shown in FIG. 9, docking station 900 includes a display 902 and a docking port 904 which is occupied by an actuator component 102 as shown in FIGS. 1 and 2. Further details regarding docking station components are provided in International Patent Application Serial No. PCT/US2018/037157 filed Jun. 12, 2018; the disclosure of which is herein incorporated by reference.

### Smart Device Configurations

As described above, in some instances the therapeutic agent delivery system includes an identifier and the actuator component includes an identifier reader, such that the device may be viewed as a “smart” device. In such embodiments, a variety of different types of information may be stored on the identifier. Reading of the identifier by the identifier reader, e.g., when the therapeutic agent delivery component is release-ably engaged to the actuator component, transfers the information to the actuator component.

In some instances, the identifier includes therapeutic agent delivery component information. Therapeutic agent delivery component information is information or data about the therapeutic agent delivery component itself. Such information may include therapeutic agent delivery component historical information. Historical information is information about the nature of the therapeutic agent delivery component and/or one or more past events experienced by the therapeutic agent delivery component. Historical information includes, but is not limited to: a therapeutic agent identifier (e.g., the name of the therapeutic agent (or a proxy thereof) contained in the therapeutic agent delivery component), manufacturing lot number for therapeutic agent and/or therapeutic agent delivery component, therapeutic agent delivery component handling information (e.g., information about the supply channel through which the therapeutic agent delivery component has passed), therapeutic agent delivery component dose, concentration, and/or volume, and therapeutic agent delivery component expiration date, chain of custody information (e.g., shipment tracking information including time and geographical information, and temperature information over time, such as any storage temperature excursions that may have occurred, etc.) and the like. As such, historical information may include information about a particular therapeutic agent contained in the therapeutic agent delivery component. Such information may include, but is not limited to, identity of the therapeutic agent (brand name and/or generic name), date of manufacture, date of expiry, source of manufacture, dosage amount, drug concentration, intended route of administration, handling and storage information, delivery volume, indication for use, lot number, etc.

In some instances, the identifier includes therapeutic agent delivery component information that is therapeutic agent delivery component use information. Therapeutic agent delivery component use information is information or data about the actual use of the therapeutic agent delivery component, e.g., the actual employment of that therapeutic agent delivery component to deliver a therapeutic agent to a target delivery site. Such information may vary, and may include use date information (i.e., information about the data, time, etc., at which the component was used); administration information, e.g., confirmation that actual delivery to a subject occurred); identity of the subject to which the therapeutic agent was administered; condition of the subject for which the therapeutic agent was administered, etc. As will be appreciated by the skilled artisan, this information could be stored directly on the identifier, or could be looked up in a linked database using the identifier information.

Smart device embodiments, e.g., as described above, allow for one or more desirable capabilities, including but not limited to inventory management capabilities, enhanced therapeutic capabilities, medical record history capabilities, data analytics capabilities, and the like. For example, a variety of different inventory management capabilities are provided by smart device embodiments, including automated reordering of therapeutic agent delivery component by a user (e.g., according to user preset preferences), tracking of individual therapeutic agent delivery components (e.g., to manage lost, stolen, or expired goods components), and the like. A variety of different enhanced therapeutic capabilities are provided by smart device embodiments, including auto-generation of procedure notes, communication with existing electronic medical records for integration in a patient chart, sending data on drug/dose/route for documentation purposes and/or billing purposes, aggregating data on drug/dose/route for market research and analytics, facilitating documentation for reimbursement, increasing billing process efficiency, improving drug and device charge capture, transferring drug between sites, providing dosing frequency alerts, national drug code tracking, ICD code data generation, generating aging reports on unpaid invoices, assessing value of inventory on hand, generating reimbursement reports and the like. Examples of capabilities provided by smart device embodiments, e.g., as described herein, are further provided in Published United States Patent Application Publication Nos. 20160030683; 20170098058; 20170119969; 20170124284 and 20170124285; the disclosures of which capabilities are incorporated herein by references.

Data obtained as described above may be employed by a user using any convenient application, where such applications may be configured for use on any convenient computing device, e.g., a desktop device, laptop device, mobile device, etc. A mobile or non-mobile device app-based interface may be configured to enable users to easily access and interact with data. An app-based interface may be provided to patients, enabling them to have better understanding of their existing treatment regimen, including when they are due for their next treatment, total number of treatments received, and the like. This mobile or non-mobile App can be synced to other key components of the patient's medical record, including the electronic medical record and imaging software. In one embodiment, the application can display a patient's OCT picture and key metrics (for example, central subfoveal thickness, macular thickness and the like) along with data collected over time (for example, the net decrease in central subfoveal thickness following treatments). This data interface can provide key treatment information to patients, such as the name, dose, lot number, and date of medication injection, creating a real-time treatment log for patients. Based on this log, patients can have the opportunity to play a more active role in their treatment, for example calling the physician to schedule a follow up if an appointment is to be missed. In one embodiment, a designated care-giver could have access to alerts to help schedule the patient for their treatments. In another embodiment, the application can be used by a pharmaceutical company to communicate key information to patients, such discontinued lot numbers, pricing discounts, new therapies to be released, educational materials, and the like. In one embodiment, the mobile app may be configured communicate with the device base-station or handheld device to communicate key information through RFID technology, NFC technology, or the like. In another embodiment, the app may be configured for remote communications, e.g., through Bluetooth, WiFi, or cellular networks. In another embodiment, the app may be configured so that the data is only be received following generation of a single use code to protect patient information.

Examples of capabilities provided by smart device embodiments, e.g., as described herein, are further provided International Patent Application Serial No. PCT/US2018/037157 filed Jun. 12, 2018; the disclosures of which capabilities are incorporated herein by reference.

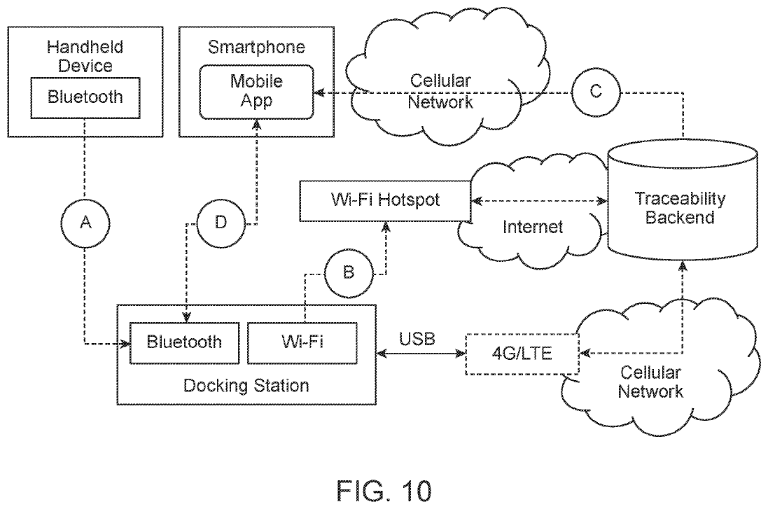

FIG. 10 provides a schematic view of a smart system according to an embodiment of the invention. As illustrated in FIG. 10, the smart system includes a handheld delivery device, a handheld computing device, such as a smartphone, a docking station, and a remote server. Data communication between these different components of the system is provided via various communication protocols, including wireless and/or wired communication products. The therapeutic delivery system includes a tip will that includes an RFID tag and the handheld actuator component includes a Bluetooth module and RFID reader. The docking station has Bluetooth and WiFi modules with a USB port. Message Queue Telemetry Transport (MQTT) and WiFi Accessory Configuration (WAC) is used to communicate between all the devices. In the illustrated system, an Internet of Things (IoT) approach to the supply chain management may be employed, e.g., to achieve one or more of the following results: authentication of the drug product tip with the device; medication validation (correct drug, expiration date); traceability and compliance (Medicare, etc.); supply chain management, such as reordering, etc., injection validation, Electronic Health Record (EHR) one way integration, etc.

### Methods

Aspects of the invention further include methods of delivering a therapeutic agent to a target tissue delivery site of a subject using therapeutic agent delivery devices of the invention. Aspects of the methods include: contacting a tissue contacting tip of a therapeutic agent delivery device, e.g., as described above, to the target tissue delivery site; actuating the pain mitigation system to mitigate pain at the target tissue delivery site; and actuating the therapeutic agent delivery system to deliver a therapeutic agent to the target tissue delivery site.

As reviewed above, the target tissue delivery site may vary. Examples of target tissue delivery sites include both external and internal delivery sites, wherein internal delivery sites include those sites located in body cavities. External sites include keratinized sites, as well as sites characterized by cutaneous membranes, mucous membranes, and tissue of the mucocutaneous zone. In some instances, the target tissue delivery site is an ocular site, where ocular sites of interest include a region that begins at the corneal limbus and extends anywhere from 1 mm to 10 mm posterior to the limbus, such as 2 mm to over 8 mm posterior to the limbus. In some instances, the area of interest includes the cornea and the corneal limbus.

To contact the distal end of the device with the target tissue site, the device may be manipulated so that the distal end of the device contacts the target tissue site. Where desired, contact of the distal end with the target tissue delivery site may be maintained by urging the distal end against the target tissue delivery site with moderate force.

The pain mitigation system is actuated to mitigate pain at the target tissue delivery site. Depending on the nature of the pain mitigation system, the pain mitigation system may be activated before or after contact of the distal end of the device with the target tissue delivery site. For example, where the pain mitigation system is a cooling system, the pain mitigation system may be activated so that a tissue engager of the cooling system is at a desired temperature prior to contact of the distal end of the device, and tissue engager, with the target tissue delivery site. Alternatively, where the pain mitigation system provides for pain mitigation via another mechanism, the pain mitigation system may be activated after contact of the distal end of the device with the target tissue delivery site.

Following pain mitigation at the target tissue delivery site, the therapeutic agent delivery system is actuated to deliver an amount, e.g., dosage, of a therapeutic agent to the target tissue delivery site. Where actuation of the therapeutic agent delivery system results in automated therapeutic agent delivery, the device is held in such a manner such that contact of the distal end of the device with the target tissue delivery site is maintained during the therapeutic agent delivery. Following active agent delivery, an injector of the therapeutic agent delivery device may be withdrawn from the target tissue delivery site, e.g., using a controlled retraction profile.

The devices may be employed to deliver a therapeutic agent to a target tissue delivery site of different types of subjects. Generally, such subjects are “mammals” or “mammalian,” where these terms are used broadly to describe organisms which are within the class mammalia, including the orders carnivore (e.g., dogs and cats), rodentia (e.g., mice, guinea pigs, and rats), and primates (e.g., humans, chimpanzees, and monkeys). In certain embodiments, the subjects are humans. The methods may be diagnostic and/or therapeutic methods.

In some instances, the methods include assembling a therapeutic agent delivery device, e.g., by operably engaging a therapeutic agent delivery system in a receiving space of an actuator component to produce a complete device, e.g., as described above. In some instances, the method includes removing the actuator component from a docking station, such as described above. In some instances, the method further includes removing the therapeutic agent delivery component from the receiving space of the actuator component. The removing may include disposing the therapeutic agent delivery system. In some instances, the method further includes docking the actuator component in the docking station.

### Utility

Devices of the invention, e.g., as described above, find use in the delivery of a variety of different types of therapeutic agents to a target tissue delivery site to treat a variety of different types of conditions. The therapeutic agent delivery devices of the invention may be used to deliver a therapeutic agent to a variety of target tissue delivery sites. Examples of target tissue delivery sites include both external and internal delivery sites, wherein internal delivery sites include those sites located in body cavities. External sites may include keratinized sites, as well as sites characterized by cutaneous membranes, mucous membranes, and tissue of the mucocutaneous zone. In some instances, the target tissue delivery site is an ocular tissue delivery site, where ocular tissue delivery sites of interest include a region that begins at the corneal limbus and extends anywhere from 2 mm to over 8 mm posterior to the limbus, such as 3 mm to 6 mm from the corneal limbus, e.g., 3 to 4 mm from the corneal limbus, e.g., to allow intraocular injection via Pars plana or Pars plicata. Ocular tissue delivery sites may include conjunctiva, episclera, and sclera of the eye. In some instances, the subject devices are used for intravitreal injection therapy (IVT), retrobulbar injection therapy, subtenon injection therapy, subretinal injection therapy, suprachoroial injection, subconjunctival injection therapy, intracameral injection therapy, and the like.

Examples of therapeutic agents that may be delivered using devices of the invention include, but are not limited to steroids such as corticosteroids including dexamethasone, fluocinolone, loteprednol, difluprednate, fluorometholone, prednisolone, medrysone, triamcinolone, betamethasone and rimexolone; nonsteroidal anti-inflammatory agents such as salicylic-, indole acetic-, aryl acetic-, aryl propionic- and enolic acid derivatives including bromfenac, diclofenac, flurbiprofen, ketorolac tromethamine and nepafenac; antibiotics including azithromycin, bacitracin, besifloxacin, ciprofloxacin, erythromycin, gatifloxacin, gentamicin, levofloxacin, moxifloxacin, ofloxacin, sulfacetamide and tobramycin; VEGF inhibitors such as tyrosine kinase inhibitors, antibodies to VEGF, antibody fragments to VEGF, VEGF binding fusion proteins; PDGF inhibitors, antibodies to PDGF, antibody fragments to PDGF, PDGF binding fusion proteins; anti-TNF alpha agents such as TNF-alpha binding agents, including antibodies to TNF-alpha, antibody fragments to TNF-alpha and TNF-alpha binding fusion proteins including infliximab, etanercept, adalimumab, certolizumab and golimumab; mTOR inhibitors such as sirolimus, sirolimus analogues, Everolimus, Temsirolimus and mTOR kinase inhibitors; cells such as mesenchymal cells (e.g. mesenchymal stem cells), or cells transfected to produce a therapeutic compound; neuroprotective agents such as antioxidants, calcineurin inhibitors, NOS inhibitors, sigma-1 modulators, AMPA antagonists, calcium channel blockers and histone-deacetylases inhibitors; antihypertensive agents such as prostaglandin analogs, beta blockers, alpha agonists, and carbonic anhydrase inhibitors; aminosterols such as squalamine; antihistamines such as H 1-receptor antagonists and histamine H2-receptor antagonists; therapeutic cells; tyrosine kinase inhibitors and nucleic acid based therapeutics such as gene vectors, complement system modulators, e.g., inhibitors; chemotherapeutic agents; insulin; cytokines, e.g., interferon beta 1-alpha; nucleic acid active agents, e.g., plasmids and siRNA; interleukin-4 receptor antagonists (such as Dupilumab etc.), interleukin-6 receptor antagonists (such as Sarlumab, etc.), proprotein convertase subtilisin kexin type 9 inhibitor antibodies (such as Alirocumab), interleukin-1 inhibitors (such as Rilonacept), and PD-1 checkpoint inhibitors (such as Cemiplimab), monoclonal antibodies targeting HER2 (such as trastuzumab), monoclonal antibodies that target CD52 (such as Alemtuzumab), RANK/RANKL inhibition (such as denosumab), antibodies against CD20 (such as rituximab and veltuzumab), and antibodies directed against CA125 (such as abagovomab); etc.

In some instances, the active agent composition is a vaccine composition. As described herein, a vaccine composition is a composition that includes one or more immunogens for vaccinating a mammal (e.g., a dog, cat, horse, sheep, pig, cow, heifer, calf, steer, bull, goat, llama, ferret, or human), a bird (e.g., a chicken and turkey), or a fish (e.g., a trout, salmon, or sea bass). For example, a vaccine composition can include the immunogen or immunogens or vaccine antigen or antigens of an anti-pathogen vaccine, an anti-cancer vaccine, or an immunocontraception vaccine. Examples of immunogens of an anti-pathogen vaccine that can be formulated into a vaccine composition and used as described herein include, without limitation, antigens (e.g., hemagglutinin, neuraminidases, glycoproteins, or nucleoproteins) from viruses such as dengue, HIV, influenza, HPV, HSV, HZV, feline panleukopenia virus, feline infectious peritonitis virus, rabies, porcine reproductive and respiratory disease virus (PRRS), bursal disease virus, caprine arthritis and encephalitis virus, and hepatitis virus, antigens (e.g., F1-V, PspA, rPA) from bacteria such as Mycobacterium tuberculosis, Clostridium tetani (e.g., tetanus), Mycobacterium avium paratuberculosis, Anaplasma, Borrelia, Leptospira, Ehrlichia, Brucella, Vibrio, and Aeromonas, antigens from fungal organism such as Cryptococcus, Histoplasma, Pneumocystis, and Aspergillus, antigens from protozoans such as Plasmodium (malaria), Leishmania, Babesia, Eimeria, and Icthyopthiris multifiliis, and antigens from nematodes such as Ascaris, Ancyclostoma, Necator, Oesophagostomum, and Haemonchus. Examples of immunogens of an anti-cancer vaccine that can be formulated into a vaccine depot and used as described herein include, without limitation, cancer antigens such as MUC-1, CA-125, Prostate serum antigen, and mesothelin. Examples of immunogens of an immunocontraception vaccine that can be formulated into a vaccine composition and used as described herein include, without limitation, antigens such as gonadotropin-releasing hormone, gonadotropin-releasing hormone linked to carrier proteins such as the mollusk hemocyanins blue protein, keyhole limpet hemocyanian, or ovalbumin, luteinizing hormone, luteinizing hormone beta subunit, and porcine zona pellucida. In some cases, a vaccine depot can include a multiple antigenic peptide of GnRH dimer as described elsewhere (Beekman et al., Vaccine, 17:2043-2050 (1999)). A vaccine composition can include any appropriate amount of an immunogen. For example, a vaccine depot can be formulated to include between about 500 ng and about 1 mg by weight of an immunogen of, for example, about 10,000 and 100,000 MW (e.g., about 18,000 MW).

The device may be employed to deliver a therapeutic agent to treat a variety of different disease conditions. Disease conditions of interest include, but are not limited to, ocular conditions, such as ocular disease conditions, such as intraocular neovascular disease conditions. An “intraocular neovascular disease” is a disease characterized by ocular neovascularisation. Examples of intraocular neovascular diseases include, for example, proliferative retinopathies, choroidal neovascularization (CNV), age-related macular degeneration (AMD), geographic atrophy (GA), diabetic and other ischemia-related retinopathies, diabetic macular edema, pathological myopia, von Hippel-Lindau disease, histoplasmosis of the eye, Central Retinal Vein Occlusion (CRVO), Branch Retinal Vein Occlusion (BRVO), pterygium, corneal neovascularization, and retinal neovascularization. The term “age-related macular degeneration” refers to a medical condition which usually affects older adults and results in a loss of vision in the center of the visual field (the macula) because of damage to the retina. Some or all of these conditions can be treated by intravitreal injection of a VEGF-antagonist, e.g., as described above. Other ocular conditions that may be treated in accordance with aspects of the invention include, but are not limited to: retinal detachments (pneumatic retinopexy), by using devices of the invention to inject a gas into the eye, where the device may control the depth of injection to a desired/optimal depth. Disease conditions of interest also include central serous chorioretinopathy and uveitis, including anterior uveitis, Pars planitis, intermediate uveitis, and posterior uveitis.

### Kits

Also provided are kits that include at least one or more therapeutic agent delivery components, e.g., as described above. For example, a kit may include a needle and a tissue contacting tip, e.g., as described above, where these components may be separate or operably engaged with each other as a composite structure. A kit may further include, where desired, an actuator component, a docking station, etc. The kit components may be present in packaging, which packaging may be sterile, as desired.

Also present in the kit may be instructions for using the kit components. The instructions may be recorded on a suitable recording medium. For example, the instructions may be printed on a substrate, such as paper or plastic, etc. As such, the instructions may be present in the kits as a package insert, in the labeling of the container of the kit or components thereof (i.e. associated with the packaging or subpackaging) etc. In other embodiments, the instructions are present as an electronic storage data file present on a suitable computer readable storage medium, e.g., portable flash drive, DVD- or CD-ROM, etc. The instructions may take any form, including complete instructions for how to use the device or as a website address with which instructions posted on the world wide web may be accessed.

Notwithstanding the appended clauses, the disclosure set forth herein is also defined by the following clauses:

1. A therapeutic agent delivery device for delivering a therapeutic agent to a target tissue delivery site, the device comprising:
(a) a therapeutic agent delivery system comprising:
   (i) a drug container having a proximal and distal end and comprising a liquid active agent composition;
   (ii) a needle having a proximal and distal end, where the proximal end is operatively coupled to the distal end of the drug container; and
   (iii) a tissue contacting tip operatively coupled to the needle; and
(b) an actuator component comprising:
   (i) a therapeutic agent delivery system receiving space containing the therapeutic agent delivery system;
   (ii) an actuator configured to actuate the therapeutic agent delivery system;
   (iii) a pain mitigation system operatively coupled to the tissue contacting tip and configured to mitigate pain at the target tissue delivery site.
      2. The therapeutic agent delivery device according to Clause 1, wherein the drug container has a volume ranging from 0.10 to 5.0 cc.
      3. The therapeutic agent delivery device according to Clause 2, wherein the drug container has a volume ranging from 0.25 to 1.50 cc.
      4. The therapeutic agent delivery device according to Clause 3, wherein the drug container has a volume ranging from 0.50 to 1.0 cc.
      5. The therapeutic agent delivery device according to Clause 4, wherein the drug container has a volume ranging from 0.70 to 0.80 cc.
      6. The therapeutic agent delivery device according to any of the preceding clauses, wherein the drug container is a glass drug container.
      7. The therapeutic agent delivery device according to any of Clauses 1 to 5, wherein the drug container is a polymeric drug container.
      8. The therapeutic agent delivery device according to any of the preceding clauses, wherein the needle has a gauge ranging from 27 to 35.
      9. The therapeutic agent delivery device according to Clause 8, wherein the needle has a gauge ranging from 30 to 33.
      10. The therapeutic agent delivery device according to any of the preceding clauses, wherein the proximal end of the needle is coupled to the distal end of the drug container by a luer fitting.
      11. The therapeutic agent delivery device according to any of the preceding clauses, wherein the tissue contacting tip comprises a proximal end attached to the needle and a distal end extending beyond the distal end of the needle by a distance ranging from 2 to 20 mm.
      12. The therapeutic agent delivery device according to Clause 11, wherein the distal end of the tissue contacting tip comprises a passageway configured to provide for passage of the distal end of the needle.
      13. The therapeutic agent delivery device according to Clause 12, wherein the distal end of the needle moves relative to the distal end of the tissue contacting tip upon actuation of the actuator.
      14. The therapeutic agent delivery device according to any of Clauses 11 to 13, wherein the distal end of the tissue contacting tip comprises a thermally conductive material.
      15. The therapeutic agent delivery device according to any of the preceding clauses, wherein the liquid active agent composition comprises a VEGF modulator.
      16. The therapeutic agent delivery device according to Clause 15, wherein the VEGF modulator comprises an antibody or binding fragment thereof.
      17. The therapeutic agent delivery device according to any of Clauses 1 to 14, wherein the liquid active agent composition comprises a complement system modulator.
      18. The therapeutic agent delivery device according to Clause 17, wherein the complement system modulator comprises a small molecule, antibody or binding fragment thereof.
      19. The therapeutic agent delivery device according to any of Clauses 1 to 14, wherein the liquid active agent comprises an anti-TNF-alpha agent.
      20. The therapeutic agent delivery device according to Clause 19, wherein the anti-TNF-alpha agent is a TNF-alpha binding agent.
      21. The therapeutic agent delivery device according to Clause 20, wherein the TNF-alpha binding agent is an antibody to TNF-alpha.
      22. The therapeutic agent delivery device according to Clause 21, wherein the antibody to TNF-alpha is adalimumab.
      23. The therapeutic agent delivery device according to Clause 20, wherein the TNF-alpha binding agent is a TNF-alpha binding fusion protein.
      24. The therapeutic agent delivery device according to Clause 23, wherein the TNF-alpha binding fusion protein is etanercept.
      25. The therapeutic agent delivery device according to any of Clauses 1 to 14, wherein the liquid active agent comprises a vaccine composition.
      26. The therapeutic agent delivery device according to any of the preceding clauses, wherein the pain mitigation system comprises an anesthesia producing system.
      27. The therapeutic agent delivery device according to Clause 26, wherein the anesthesia producing system comprises a cooling system.
      28. The therapeutic agent delivery device according to Clause 27, wherein the cooling system comprises a thermoelectric cooling system.
      29. The therapeutic agent delivery device according to Clause 28, wherein the thermoelectric cooling system comprises a Peltier unit, a conductor coupling the Peltier unit to the tissue contacting tip and a heat sink coupled to the Peltier unit.
      30. The therapeutic agent delivery device according to Clause 29, wherein the heat sink comprises a liquid.
      31. The therapeutic agent delivery device according to Clause 29, wherein the heat sink is a solid.
      32. The therapeutic agent delivery device according to Clause 27, wherein the cooling system comprises a substance having a freezing temperature of 0° C. or lower.
      33. The therapeutic agent delivery device according to any of the preceding clauses, wherein the actuator comprises a motor.
      34. The therapeutic agent delivery device according to Clause 33, wherein the actuator comprises more than one motor.
      35. The therapeutic agent delivery device according to Clause 34, wherein each motor is unidirectional.
      36. The therapeutic agent delivery device according to any of Clauses 1 to 32, wherein the actuator comprises a spring.
      37. The therapeutic agent delivery device according to any of Clauses 1 to 32, wherein the actuator comprises a manual actuator.
      38. The therapeutic agent delivery device according to any of the preceding clauses, wherein the actuator is configured to sequentially move the drug container in a first priming motion and a second injection motion.
      39. The therapeutic agent delivery device according to Clause 38, wherein the actuator is further configured to withdraw the needle back into the device following injection of the active agent composition.
      40. The therapeutic agent delivery device according to Clause 39, wherein the actuator comprises a first motor configured to sequentially move the drug container in a first priming motion and a second injection motion and a second motor configured to withdraw the needle back into the device following injection of the active agent composition.
      41. The therapeutic agent delivery device according to any of the preceding clauses, wherein the therapeutic agent delivery system comprises an identifier and the actuator component comprises an identifier reader.
      42. The therapeutic agent delivery device according to Clause 41, wherein the device is configured to be active only when the identifier reader detects an acceptable therapeutic agent delivery system identifier.
      43. The therapeutic agent delivery device according to any of the preceding clauses, wherein the device further comprises a communications module.
      44. The therapeutic agent delivery device according to Clause 43, wherein the communications module is configured for wireless communication.
      45. The therapeutic agent delivery device according to any of the preceding clauses, wherein the device is a handheld device.
      46. The therapeutic agent delivery device according to any of the preceding clauses, wherein the actuator component further comprises a display.
      47. The therapeutic agent delivery device according to any of the preceding clauses, wherein the target tissue delivery site comprises an ocular tissue delivery site.
      48. A therapeutic agent delivery device actuator component, the actuator component comprising:
      (a) a therapeutic agent delivery system receiving space configured to receive a therapeutic agent delivery system comprising:
(i) a drug container having a proximal and distal end and comprising a liquid active agent composition;
(ii) a needle having a proximal and distal end, where the
   proximal end is operatively coupled to the distal end of the drug container; and
(iii) a tissue contacting tip operatively coupled to the needle;
   (b) an actuator configured to actuate the therapeutic agent delivery system; and
   (c) a pain mitigation system operatively configured to operative couple to the tissue contacting tip and configured to mitigate pain at a target tissue delivery site.
   49. The therapeutic agent delivery device actuator component according to Clause 48, wherein the pain mitigation system comprises an anesthesia producing system.
   50. The therapeutic agent delivery device actuator component according to Clause 49, wherein the anesthesia producing system comprises a cooling system.
   51. The therapeutic agent delivery device actuator component according to Clause 50, wherein the cooling system comprises a thermoelectric cooling system.
   52. The therapeutic agent delivery device actuator component according to Clause 51, wherein the thermoelectric cooling system comprises a Peltier unit, a conductor coupling the Peltier unit to the tissue contacting tip and a heat sink coupled to the Peltier unit.
   53. The therapeutic agent delivery device actuator component according to Clause 52, wherein the heat sink comprises a liquid.
   54. The therapeutic agent delivery device actuator component according to Clause 52, wherein the heat sink is a solid.
   55. The therapeutic agent delivery device actuator component according to Clause 50, wherein the cooling system comprises a substance having a freezing temperature of 0° C. or lower.
   56. The therapeutic agent delivery device actuator component according to any of Clauses 48 to 55, wherein the actuator comprises a motor.
   57. The therapeutic agent delivery device actuator component according to Clause 56, wherein the actuator comprises more than one motor.
   58. The therapeutic agent delivery device actuator component according to Clause 57, wherein each motor is unidirectional.
   59. The therapeutic agent delivery device actuator component according to any of Clauses 48 to 55, wherein the actuator comprises a spring.
   60. The therapeutic agent delivery device actuator component according to any of Clauses 48 to 55, wherein the actuator comprises a manual actuator.
   61. The therapeutic agent delivery device actuator component according to any of Clauses 48 to 60, wherein the actuator is configured to sequentially move the drug container in a first priming motion and a second injection motion.
   62. The therapeutic agent delivery device actuator component according to Clause 61, wherein the actuator is further configured to withdraw the needle back into the device following injection of the active agent composition.
   63. The therapeutic agent delivery device actuator component according to Clause 62, wherein the actuator comprises a first motor configured to sequentially move the drug container in a first priming motion and a second injection motion and a second motor configured to withdraw the needle back into the device following injection of the active agent composition.
   64. The therapeutic agent delivery device actuator component according to any of Clauses 48 to 63, wherein the actuator component comprises an identifier reader configured to read an identifier on a therapeutic agent delivery system.
   65. The therapeutic agent delivery device actuator component according to Clause 64, wherein the actuator component is configured to be active only when the identifier reader detects an acceptable therapeutic agent delivery system identifier.
   66. The therapeutic agent delivery device actuator component according to any of Clauses 48 to 65, wherein the device further comprises a communications module.
   67. The therapeutic agent delivery device actuator component according to Clause 66, wherein the communications module is configured for wireless communication.
   68. The therapeutic agent delivery device according to any of the preceding clauses, wherein the actuator component further comprises a display.
   69. The therapeutic agent delivery device actuator component according to any of Clauses 48 to 68, wherein the actuator component is configured to be handheld.
   70. A composite structure comprising:
(a) a needle having a proximal and distal end, where the proximal end is configured to operatively couple to a drug container; and
(b) a tissue contacting tip operatively coupled to the needle;
wherein the tissue contacting tip comprises a proximal end attached to the needle and a distal end extending beyond the distal end of the needle by a distance ranging from 2 to 20 mm.
   71. The composite structure according to Clause 70, wherein the distal end of the tissue contacting tip comprises a passageway configured to provide for passage of the distal end of the needle.
   72. The composite structure according to any of Clauses 70 to 71, wherein a locking mechanism maintains the distal end of the needle relative to the distal end of the tissue contacting tip.
   73. The composite structure according to Clause 72, wherein the locking mechanism is releasable upon movement of the tissue contacting tip relative to the needle.
   74. The composite structure according to Clause 73, wherein the movement comprises rotational movement.
   75. The composite structure according to any of Clauses 70 to 74, wherein the distal end of the tissue contacting tip comprises a thermally conductive material.
   76. The composite structure according to any of Clauses 70 to 75, wherein the proximal end of the needle comprises a luer fitting.
   77. A therapeutic agent delivery system comprising:
(a) a drug container having a proximal and distal end and comprising a liquid active agent composition;
(b) a needle having a proximal and distal end, where the proximal end is operatively coupled to the distal end of the drug container; and
(c) a tissue contacting tip operatively coupled to the needle.
   78. The therapeutic agent delivery system according to Clause 77, wherein the drug container has a volume ranging from 0.10 to 5.0 cc.
   79. The therapeutic agent delivery system according to Clause 78, wherein the drug container has a volume ranging from 0.25 to 1.50 cc.
   80. The therapeutic agent delivery system according to Clause 79, wherein the drug container has a volume ranging from 0.50 to 1.0 cc.
   81. The therapeutic agent delivery system according to Clause 80, wherein the drug container has a volume ranging from 0.70 to 0.80 cc.
   82. The therapeutic agent delivery system according to any of Clauses 77 to 81, wherein the drug container is a glass drug container.
   83. The therapeutic agent delivery system according to any of Clauses 77 to 81, wherein the drug container is a polymeric drug container.
   84. The therapeutic agent delivery system according to any of Clauses 77 to 83, wherein the needle has a gauge ranging from 27 to 35.
   85. The therapeutic agent delivery system according to Clause 84, wherein the needle has a gauge ranging from 30 to 33.
   86. The therapeutic agent delivery system according to any of Clauses 77 to 85, wherein the proximal end of the needle is coupled to the distal end of the drug container by a luer fitting.
   87. The therapeutic agent delivery system according to any of Clauses 77 to 86, wherein the tissue contacting tip comprises a proximal end attached to the needle and a distal end extending beyond the distal end of the needle by a distance ranging from 2 to 20 mm.
   88. The therapeutic agent delivery system according to Clause 87, wherein the distal end of the tissue contacting tip comprises a passageway configured to provide for passage of the distal end of the needle.
   89. The therapeutic agent delivery system according to Clause 88, wherein a locking mechanism maintains the distal end of the needle relative to the distal end of the tissue contacting tip.
   90. The therapeutic agent delivery system according to Clause 89, wherein the locking mechanism is releasable upon movement of the tissue contacting tip relative to the needle.
   91. The therapeutic agent delivery system according to Clause 90, wherein the movement comprises rotational movement.
   92. The therapeutic agent delivery system according to any of Clauses 87 to 91, wherein the distal end of the tissue contacting tip comprises a thermally conductive material.
   93. The therapeutic agent delivery system according to any of Clauses 77 to 92, wherein the liquid active agent composition comprises a VEGF modulator, such as an antibody or binding fragment thereof.
   94. The therapeutic agent delivery system according to any of Clauses 77 to 92, wherein the liquid active agent composition comprises a complement system modulator.
   95. The therapeutic agent delivery system according to Clause 94, wherein the complement system modulator comprises a small molecule, antibody or binding fragment thereof.
   96. The therapeutic agent delivery system according to any of Clauses 77 to 92, wherein the liquid active agent comprises an anti-TNF-alpha agent.
   97. The therapeutic agent delivery system according to Clause 96, wherein the anti-TNF-alpha agent is a TNF-alpha binding agent.
   98. The therapeutic agent delivery system according to Clause 97, wherein the TNF-alpha binding agent is an antibody to TNF-alpha.
   99. The therapeutic agent delivery device according to Clause 98, wherein the antibody to TNF-alpha is adalimumab.
   100. The therapeutic agent delivery device according to Clause 97, wherein the TNF-alpha binding agent is a TNF-alpha binding fusion protein.
   101. The therapeutic agent delivery device according to Clause 100, wherein the TNF-alpha binding fusion protein is etanercept.
   102. The therapeutic agent delivery device according to any of Clauses 77 to 92, wherein the liquid active agent comprises a vaccine composition.
   103. A method of delivering a therapeutic agent to a target tissue delivery site, the method comprising:
(A) contacting a tissue contacting tip of therapeutic agent delivery device to the target tissue delivery site, wherein the therapeutic agent delivery device comprises:
(1) a therapeutic agent delivery system comprising:
   (a) a drug container having a proximal and distal end and comprising a liquid active agent composition;
   (b) a needle having a proximal and distal end, where the proximal end is operatively coupled to the distal end of the drug container; and
   (c) a tissue contacting tip operatively coupled to the needle; and
(2) an actuator component comprising:
   (a) a therapeutic agent delivery system receiving space containing the therapeutic agent delivery system;
   (b) an actuator configured to actuate the therapeutic agent delivery system;
   (c) a pain mitigation system operatively coupled to the tissue contacting tip and configured to mitigate pain at the target tissue delivery site;
(B) actuating the pain mitigation system to mitigate pain at the target tissue delivery site; and
(C) actuating the therapeutic agent delivery system to deliver a therapeutic agent to the target tissue delivery site.
   104. The method according to Clause 103, wherein the drug container has a volume ranging from 0.10 to 5.0 cc.
   105. The method according to Clause 104, wherein the drug container has a volume ranging from 0.25 to 1.50 cc.
   106. The method according to Clause 105, wherein the drug container has a volume ranging from 0.50 to 1.0 cc.
   107. The method according to Clause 106, wherein the drug container has a volume ranging from 0.70 to 0.80 cc.
   108. The method according to any of Clauses 103 to 107, wherein the drug container is a glass drug container.
   109. The method according to any of Clauses 103 to 107, wherein the drug container is a polymeric drug container.
   110. The method according to any of Clauses 103 to 109, wherein the needle has a gauge ranging from 27 to 35.
   111. The method according to Clause 110, wherein the needle has a gauge ranging from 30 to 33.
   112. The method according to any of Clauses 103 to 111, wherein the proximal end of the needle is coupled to the distal end of the drug container by a luer fitting.
   113. The method according to any of Clauses 103 to 112, wherein the tissue contacting tip comprises a proximal end attached to the needle and a distal end extending beyond the distal end of the needle by a distance ranging from 2 to 20 mm.
   114. The method according to Clause 113, wherein the distal end of the tissue contacting tip comprises a passageway configured to provide for passage of the distal end of the needle.
   115. The method according to Clause 114, wherein the distal end of the needle moves relative to the distal end of the tissue contacting tip upon actuation of the actuator.
   116. The method according to any of Clauses 113 to 115, wherein the distal end of the tissue contacting tip comprises a thermally conductive material.
   117. The method according to any of Clauses 103 to 116, wherein the liquid active agent composition comprises a VEGF modulator.
   118. The method according to Clause 117, wherein the VEGF modulator comprises an antibody or binding fragment thereof.
   119. The method according to any of Clauses 103 to 116, wherein the liquid active agent composition comprises a complement system modulator.
   120. The method according to Clause 119, wherein the complement system modulator comprises a small molecule, antibody or binding fragment thereof.
   121. The method according to any of Clauses 103 to 120, wherein the liquid active agent comprises an anti-TNF-alpha agent.
   122. The method according to Clause 121, wherein the anti-TNF-alpha agent is a TNF-alpha binding agent.
   123. The method according to Clause 122, wherein the TNF-alpha binding agent is an antibody to TNF-alpha.
   124. The method according to Clause 123, wherein the antibody to TNF-alpha is adalimumab.
   125. The method according to Clause 122, wherein the TNF-alpha binding agent is a TNF-alpha binding fusion protein.
   126. The method according to Clause 125, wherein the TNF-alpha binding fusion protein is etanercept.
   127. The method according to any of Clauses 103 to 120, wherein the liquid active agent comprises a vaccine composition.
   128. The method according to any of Clauses 103 to 127, wherein the pain mitigation system comprises an anesthesia producing system, such as a cooling system.
   129. The method according to Clause 128, wherein the cooling system comprises a thermoelectric cooling system.
   130. The method according to Clause 129, wherein the thermoelectric cooling system comprises a Peltier unit, a conductor coupling the Peltier unit to the tissue contacting tip and a heat sink coupled to the Peltier unit.
   131. The method according to Clause 130, wherein the heat sink comprises a liquid.
   132. The method according to Clause 130, wherein the heat sink is a solid.
   133. The method according to Clause 128, wherein the cooling system comprises a substance having a freezing temperature of 0° C. or lower.
   134. The method according to any of Clauses 103 to 133, wherein the actuator comprises a motor.
   135. The method according to Clause 134, wherein the actuator comprises more than one motor.
   136. The method according to Clause 135, wherein each motor is unidirectional.
   137. The method according to any of Clauses 103 to 133, wherein the actuator comprises a spring.
   138. The method according to any of Clauses 103 to 133, wherein the actuator comprises a manual actuator.
   139. The method according to any of Clauses 103 to 138, wherein the actuator is configured to sequentially move the drug container in a first priming motion and a second injection motion.
   140. The method according to Clause 139, wherein the actuator is further configured to withdraw the needle back into the device following injection of the active agent composition.
   141. The method according to Clause 140, wherein the actuator comprises a first motor configured to sequentially move the drug container in a first priming motion and a second injection motion and a second motor configured to withdraw the needle back into the device following injection of the active agent composition.
   142. The method according to any of Clauses 103 to 141, wherein the therapeutic agent delivery system comprises an identifier and the actuator component comprises an identifier reader.
   143. The method according to Clause 142, wherein the device is configured to be active only when the identifier reader detects an acceptable therapeutic agent delivery system identifier.
   144. The method according to any of Clauses 103 to 143, wherein the device further comprises a communications module.
   145. The method according to Clause 144, wherein the communications module is configured for wireless communication.
   146. The method according to any of Clauses 103 to 145, wherein the device is a handheld device.
   147. The method according to any of Clauses 103 to 146, wherein the actuator component further comprises a display.
   148. The method according to Clause 147, wherein the method comprises reading the display.
   149. The method according to any of Clauses 103 to 148, wherein the method further comprises operably positioning the therapeutic agent delivery system in the therapeutic agent delivery system receiving space.
   150. The method according to any of Clauses 103 to 149, wherein the method further comprises assembling the therapeutic agent delivery system.
   151. The method according to any of Clauses 103 to 150, wherein the target tissue delivery site comprises an ocular tissue delivery site.
   152. The method according to Clause 151, wherein the method is a method of treating a subject for an ocular disease.
   153. A kit comprising:
   (a) a composition structure comprising:
(i) a needle having a proximal and distal end, where the proximal end is configured to operatively couple to a drug container; and
(ii) a tissue contacting tip operatively coupled to the needle; and
   (b) a drug container comprising a liquid active agent composition.
   154. The kit according to Clause 153, wherein the composite structure is a composite structure according to any of Clauses 70 to 76.
   155. The kit according to any of Clauses 153 to 154, wherein the drug container has a volume ranging from 0.10 to 5.0 cc, such as a volume ranging from 0.25 to 1.50 cc.
   156. The kit according to Clause 155, wherein the drug container has a volume ranging from 0.50 to 1.0 cc.
   157. The kit according to Clause 156, wherein the drug container has a volume ranging from 0.70 to 0.80 cc.
   158. The kit according to any of Clauses 153 to 157, wherein the drug container is a glass drug container.
   159. The kit according to any of Clauses 153 to 157, wherein the drug container is a polymeric drug container.
   160. The kit according to any of Clauses 153 to 159, wherein the liquid active agent composition comprises a VEGF modulator.
   161. The kit according to Clause 160, wherein the VEGF modulator comprises an antibody or binding fragment thereof.
   162. The kit according to any of Clauses 153 to 159, wherein the liquid active agent composition comprises a complement system modulator.
   163. The kit according to Clause 162, wherein the complement system modulator comprises a small molecule, antibody or binding fragment thereof.
   164. The kit according to any of Clauses 153 to 159, wherein the liquid active agent comprises an anti-TNF-alpha agent.
   165. The kit according to Clause 164, wherein the anti-TNF-alpha agent is a TNF-alpha binding agent.
   166. The kit according to Clause 165, wherein the TNF-alpha binding agent is an antibody to TNF-alpha.
   167. The kit according to Clause 166, wherein the antibody to TNF-alpha is adalimumab.
   168. The kit according to Clause 165, wherein the TNF-alpha binding agent is a TNF-alpha binding fusion protein.
   169. The kit according to Clause 168, wherein the TNF-alpha binding fusion protein is etanercept.
   170. The kit according to any of Clauses 153 to 159, wherein the liquid active agent comprises a vaccine composition.
   171. The kit according to any of Clauses 153 to 170, wherein the kit further comprises a therapeutic agent delivery device actuator component according to any of Clauses 48 to 69.

Embodiments of the invention provide for a number of advantages in the area of active agent delivery, e.g., to a target ocular location, including but not limited to: reduced patient pain/discomfort, more accurate active agent delivery, better tracking of dosages, easier administration, etc., as compared to prior art methods of delivering active agents to an ocular location, such as via convention needle/syringe.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. Moreover, nothing disclosed herein is intended to be dedicated to the public regardless of whether such disclosure is explicitly recited in the claims.

The scope of the present invention, therefore, is not intended to be limited to the exemplary embodiments shown and described herein. Rather, the scope and spirit of present invention is embodied by the appended claims. In the claims, 35 U.S.C. § 112(f) or 35 U.S.C. § 112(6) is expressly defined as being invoked for a limitation in the claim only when the exact phrase “means for” or the exact phrase “step for” is recited at the beginning of such limitation in the claim; if such exact phrase is not used in a limitation in the claim, then 35 U.S.C. § 112 (f) or 35 U.S.C. § 112(6) is not invoked.

What is claimed is:

1. A therapeutic agent delivery device for delivering a therapeutic agent to a target tissue delivery site, the device comprising:
(a) a therapeutic agent delivery system comprising:
(i) a drug container having a proximal and distal end and comprising a liquid active agent composition;
(ii) a needle having a proximal and distal end, where the proximal end is operatively coupled to the distal end of the drug container; and
(iii) a tissue contacting tip operatively coupled to the needle; and
(b) an actuator component comprising:
(i) a therapeutic agent delivery system receiving space containing the therapeutic agent delivery system;
(ii) an actuator configured to actuate the therapeutic agent delivery system;
(iii) a pain mitigation system operatively coupled to the tissue contacting tip and configured to mitigate pain at the target tissue delivery site.

2. The therapeutic agent delivery device according to claim 1, wherein the drug container has a volume ranging from 0.10 to 5.0 cc.

3. The therapeutic agent delivery device according to any of the preceding claims, wherein the drug container is a glass drug container or a polymeric drug container.

4. The therapeutic agent delivery device according to any of the preceding claims, wherein the needle has a gauge ranging from 27 to 35.

5. The therapeutic agent delivery device according to any of the preceding claims, wherein the proximal end of the needle is coupled to the distal end of the drug container by a luer fitting.

6. The therapeutic agent delivery device according to any of the preceding claims, wherein the tissue contacting tip comprises a proximal end attached to the needle and a distal end extending beyond the distal end of the needle by a distance ranging from 2 to 20 mm.

7. The therapeutic agent delivery device according to claim 6, wherein the distal end of the tissue contacting tip comprises a passageway configured to provide for passage of the distal end of the needle.

8. The therapeutic agent delivery device according to claim 7, wherein the distal end of the needle moves relative to the distal end of the tissue contacting tip upon actuation of the actuator.

9. The therapeutic agent delivery device according to any of the preceding claims, wherein the liquid active agent composition comprises a VEGF modulator.

10. The therapeutic agent delivery device according to any of claims 1 to 8, wherein the liquid active agent comprises an anti-TNF-alpha agent.

11. The therapeutic agent delivery device according to any of claims 1 to 8, wherein the liquid active agent comprises a vaccine composition.

12. The therapeutic agent delivery device according to any of the preceding claims, wherein the pain mitigation system comprises an anesthesia producing system.

13. A method of delivering a therapeutic agent to a target tissue delivery site, the method comprising:
(A) contacting a tissue contacting tip of therapeutic agent delivery device to the target tissue delivery site, wherein the therapeutic agent delivery device comprises:
(1) a therapeutic agent delivery system comprising:
(a) a drug container having a proximal and distal end and comprising a liquid active agent composition;
(b) a needle having a proximal and distal end, where the proximal end is operatively coupled to the distal end of the drug container; and
(c) a tissue contacting tip operatively coupled to the needle; and
(2) an actuator component comprising:
(a) a therapeutic agent delivery system receiving space containing the therapeutic agent delivery system;
(b) an actuator configured to actuate the therapeutic agent delivery system;
(c) a pain mitigation system operatively coupled to the tissue contacting tip and configured to mitigate pain at the target tissue delivery site;
(B) actuating the pain mitigation system to mitigate pain at the target tissue delivery site; and
(C) actuating the therapeutic agent delivery system to deliver a therapeutic agent to the target tissue delivery site.

14. The method according to claim 13, wherein the method is a method of treating a subject for an ocular disease.

15. A kit comprising:
(a) a composition structure comprising:
(i) a needle having a proximal and distal end, where the proximal end is configured to operatively couple to a drug container; and
(ii) a tissue contacting tip operatively coupled to the needle; and
(b) a drug container comprising a liquid active agent composition.
